(19) United States
(12) Patent Application Publication
(10) Pub. No.: US 2026/0033613 A1
(43) Pub. Date: Feb. 5, 2026

(54) SYSTEMS AND METHODS FOR PROTECTIVE HEADWEAR WITH CUSTOMIZABLE MULTI-STRAP PAP MASK ATTACHMENT SYSTEM

(71) Applicant: Victoria B. Taiwo, Bowie, MD (US)

(72) Inventor: Victoria B. Taiwo, Bowie, MD (US)

(21) Appl. No.: 19/287,723

(22) Filed: Jul. 31, 2025

Related U.S. Application Data
(60) Provisional application No. 63/677,758, filed on Jul. 31, 2024.

(51) Int. Cl. A45D 8/40; A42B 1/041; A42B 1/22; A61M 16/06

(52) U.S. Cl. CPC A45D 8/40; A42B 1/041; A42B 1/22; A61M 16/0683

(57) ABSTRACT

Systems and techniques may generally be used for providing protective headgear for air-pressure therapy users. In one aspect, the headgear may include a cap structure adapted to cover a wearer's head, a plurality of attachment points on the cap structure, and fasteners at the attachment points that may secure a continuous positive airway pressure (CPAP) mask or other PAP headgear. The attachment points may be fixed or relocatable, and the fasteners may comprise hook-and-loop, snaps, buttons, or other releasable connectors, permitting the cap to interface with differing mask frames without modifying the mask.

### REFERENCE TO RELATED APPLICATIONS

This application claims priority from U.S. provisional application Ser. No. 63/677,758, filed Jul. 31, 2024, which is incorporated by reference.

### BACKGROUND OF THE INVENTION

### Technical Field

The present disclosure generally relates to protective head coverings and accessories used with positive airway pressure (PAP) and other positive-air-pressure therapy equipment, and more particularly to bonnet-style caps designed to be worn beneath PAP headgear in order to protect the user's hair and scalp while maintaining mask stability and comfort.

### Description of the Related Art

Existing solutions to the problem being solved often rely on conventional PAP harnesses, chin straps, skull-cap style beanies, or stand-alone hair bonnets that are not integrated with PAP headgear. These approaches may include elastic fabric caps, adhesive clips, or generic headbands intended to hold mask straps in place; however, they frequently slip during sleep, create localized pressure points, contribute to hair breakage or matting, and can compromise the mask seal or overall therapy comfort.

### SUMMARY OF THE INVENTION

In general, in a first aspect, the technologies described herein relate to a protective headgear apparatus for use with a continuous-positive-airway-pressure mask. The apparatus includes a bonnet-style cap structure sized for a wearer's head, the cap having a dual-layer wall with a low-friction, hair-protective inner liner and a high-friction textile outer shell. An adjustable perimeter band with an elastic segment and cinch element enables circumferential fit. A plurality of attachment straps, each permanently affixed at a proximal end and carrying a releasable fastener at a distal end, are dimensioned and positioned so that when wrapped over an external frame of the PAP mask and secured, the straps and high-friction shell cooperatively restrain both cap and mask against displacement while the inner liner minimizes hair friction.

In general, in a second aspect, the technologies described herein relate to a method of securing a PAP mask to a user by donning the foregoing headgear, tightening the perimeter band, selecting and wrapping appropriate attachment straps over corresponding regions of the mask frame, and engaging releasable fasteners so that the mask and cap remain stably positioned while hair-protective friction reduction is maintained.

In general, in a third aspect, the technologies described herein relate to a method of manufacturing the protective headgear by cutting complementary inner-liner and outer-shell textile panels, joining them along a peripheral seam to form a cap wall, sewing the proximal ends of multiple attachment straps into that seam, affixing releasable fasteners to distal strap ends, and orienting the straps so that, when donned and fastened over a PAP mask, they secure both mask and cap against displacement.

Embodiments of the invention may include one or more of the following features. These features may be used singly or in combination: at least five attachment straps arranged as two lateral, one crown, and two diagonal rear straps to provide 360-degree stabilization; the use of hook-and-loop, snaps, buttons, magnetic couplings, or combinations thereof as releasable fasteners; permanently sewn strap anchors; exterior stowage zones for unused straps; perimeter bands adjustable to reduce cap circumference by at least five percent; repositionable straps via hook-and-loop patches or snap-button grids; strap widths between 5 mm and 25 mm and lengths sufficient to wrap over nasal-pillow forehead bars; printed alignment indicia; machine-washable materials that withstand at least fifty laundering cycles without fastener degradation; a manufacturing step of ultrasonically bonding hook-and-loop material; specific textile pairings such as satin inner liners with polyester-cotton outer shells; a matrix of complementary anchor patches enabling later strap repositioning; cinch-ring tightening of the perimeter band; stowage of unused straps flush against the outer shell; laundering while retaining strap integrity; and repositioning of straps to accommodate changes in hair volume.

The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### BRIEF DESCRIPTION OF THE FIGURES

The appended figures depict certain features of the various aspects described herein and are not to be considered limiting of the scope of this disclosure.

FIG. 1 depicts a sleep-therapy system in which a bonnet-style protective cap with multi-strap anchors secures a positive-airway-pressure mask on a resting user, as shown in some embodiments.

FIG. 2 depicts a right-side view of the protective headgear, as shown in some embodiments.

FIG. 3 depicts a rear view of the protective headgear on a user, as shown in some embodiments.

FIG. 4 depicts a three-quarter rear perspective view of the protective headgear in a relaxed state, as shown in some embodiments.

FIG. 5 depicts a side-profile view of the protective headgear, as shown in some embodiments.

FIG. 6 depicts a posterior perspective of the assembled cap and PAP interface illustrating selective deployment of the crown strap, as shown in some embodiments.

FIG. 7 depicts a rear-oblique view of the integrated headgear and mask system, as shown in some embodiments.

FIG. 8 depicts a three-quarter elevation of the protective headgear with a repositionable attachment strap and distributed anchor patch, as shown in some embodiments.

FIGS. 9A-9C depict an operational sequence for donning the cap, routing a strap, and fastening a PAP mask frame, as shown in some embodiments.

FIG. 10 depicts a flowchart outlining a method for securing a PAP mask using the protective headgear apparatus, as shown in some embodiments.

### DETAILED DESCRIPTION

In the following detailed description, numerous specific details are set forth by way of examples to provide a thorough understanding of the relevant teachings. However, it should be apparent that the present teachings may be practiced without such details. In other examples, well-known structures, functions, methods, procedures, components, and/or circuitry have been described at a relatively high level, without detail, to avoid unnecessarily obscuring aspects of the present teachings.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements, but can also mean a singular element.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

Systems and techniques described herein may be used to overcome the limitations of traditional methods for stabilizing continuous-positive-airway-pressure masks while protecting a user's hair and scalp. Conventional PAP headgear often relies on narrow elastic straps or rigid frames that concentrate pressure at discrete contact points, permit lateral slippage as the sleeper moves, and expose hair to repeated friction. Users with textured or voluminous hair frequently experience breakage, matting, and discomfort, which may lead to interrupted therapy or premature removal of the mask. Existing aftermarket bonnets or beanies are typically separate from the mask frame; when worn beneath standard headgear, such items may introduce bulk, promote shifting of the mask seal, or require ad-hoc fastening solutions that lack repeatable positioning.

To address these issues, the present disclosure provides protective headwear that may integrate a multi-strap attachment architecture with a textile cap configured for wear beneath positive-air-pressure mask frames. The headwear may include a dual-layer wall in which a low-friction liner contacts the hair and a higher-friction outer shell cooperates with mask components. A set of attachment straps, which may be permanently affixed or repositionable, may wrap over selected areas of a PAP frame and engage by way of releasable fasteners. An adjustable perimeter band may permit the cap to conform to varying head sizes and hair volumes such that both cap and mask remain in a stable orientation for the duration of a sleep session.

An example technique may include providing a bonnet-style cap sized to fit a wearer's head, the cap having a smooth inner liner that may reduce snagging on hair and a textured exterior that may resist sliding against mask materials. A plurality of attachment straps may be sewn or otherwise fixed to the cap at proximal ends and may terminate in hook-and-loop fasteners or similar releasable fasteners at distal ends. When the wearer dons the cap and draws a cinch element on an elastic perimeter band, the user may route the distal ends of the straps over corresponding regions of a PAP mask frame. Securing the fasteners may cause the straps and the high-friction outer shell to cooperate so that the mask frame and cap remain substantially stationary relative to the wearer's head while the inner liner limits friction at the hair interface.

Consider a community-health volunteer who assists unhoused individuals by setting up portable sleep-apnea stations in temporary shelters. At intake, the volunteer may fit each participant with a reusable cap that has been laundered in accordance with hygiene protocols. After the participant places the cap over their hair, the volunteer may draw the perimeter band until the cap comfortably conforms to the head contour. Observing that the participant uses a nasal-pillow PAP mask, the volunteer may select two lateral straps, one crown strap, and one rear-diagonal strap while leaving an extra strap stowed against the cap's exterior. The volunteer may wrap each chosen strap over the corresponding bar or frame element of the mask and press the hook-and-loop fasteners into engagement. Once the PAP blower starts, the mask remains seated despite turns on a cot, and the low-friction liner prevents tangling of long hair. The next morning, the straps may be detached, the cap removed, and both items may be sanitized for subsequent use with another participant.

Systems and Methods for Protective Headwear with Customizable Multi-Strap PAP Mask Attachment System

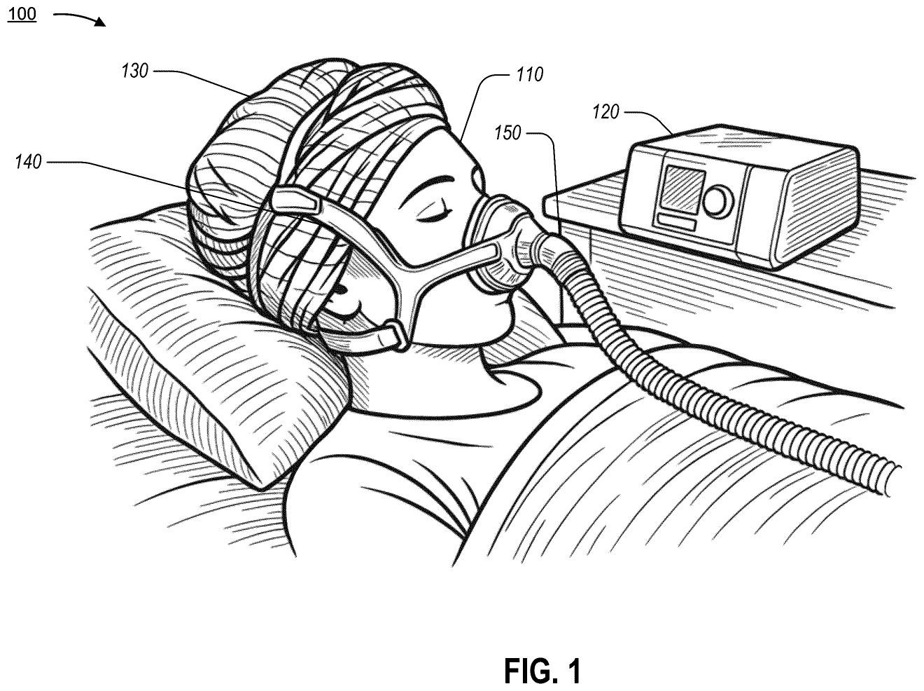

FIG. 1 depicts a sleep-therapy system in which a bonnet-style protective cap with multi-strap anchors secures a positive-airway-pressure mask on a resting user, as shown in some embodiments.

Referring to FIG. 1, sleep-therapy system 100 may include a positive-airway-pressure (PAP)

device 120 positioned on a bedside surface and configured to deliver a flow of pressurized breathing gas through an air-delivery hose 150 toward a positive-airway-pressure interface, such as a PAP mask 160 positioned over a recumbent user 110. In certain aspects, the air-delivery hose 150 may exhibit a helically ribbed wall that imparts kink resistance while maintaining a lightweight, highly flexible structure; wall thickness, polymer selection, and rib pitch may be tuned so that static bend radius remains below approximately 50 mm, for example. The hose may couple to an outlet connector on the PAP device 120 via a swivel fitting that permits rotational freedom, thereby reducing torsional loading that could otherwise translate to the mask frame.

In some aspects, PAP device 120 may house a blower, humidifier module, user interface, and wireless compliance telemetry circuitry, although these internal features are not expressly illustrated. The device may include firmware that recognizes obstructive-sleep-apnea events and may automatically adjust pressure or alarm conditions; however, these operational details are optional and are not required for cooperation with the protective headgear described below. The outlet connector of the PAP device 120 may include a standard ISO 5356-1 22-mm male taper that mates with a complementary female fitting on hose 150 so that commercially available hoses can be substituted without modification.

In various aspects, a protective cap 130 may be worn on the scalp of user 110 and may cooperate with a plurality of attachment straps 140 to restrain the PAP mask 160 against displacement. The protective cap 130 may include a cap structure formed by joining a first textile panel and a second textile panel along a peripheral seam so that an interior surface defines a low-friction, hair-protective inner liner while an exterior surface defines a higher-friction textile outer shell. For example, the inner liner may include satin, silk, or a cooling-polymer knit having a coefficient of friction against keratin fibers below about 0.25, whereas the outer shell may include a polyester-cotton blend that increases frictional interaction with a plastic or silicone mask frame. A dual-layer wall thickness may range between 0.4 mm and 1.2 mm and may remain breathable by incorporating micro-perforations or moisture-wicking channels that transport perspiration away from the scalp.

In certain aspects, the protective cap 130 may further include an adjustable perimeter band that circumscribes a lower edge of the cap structure. The band may include an elastic segment and a cinch element that can be grasped with one hand to reduce the cap circumference by at least five percent relative to an un-cinched state. The cinch element may take the form of a spring-biased cord lock, a releasable cam buckle, or a low-profile sliding ring formed of acetal or stainless-steel wire. In use, tightening the perimeter band may allow the cap to conform to different head sizes and hair volumes while distributing tension evenly so that no discrete pressure points form along the hairline of user 110.

In several aspects, the plurality of attachment straps 140 may include two lateral straps, one crown strap, and two rear-diagonal straps so that a total of five elongate straps provide 360-degree stabilization of the PAP mask 160. Each attachment strap 140 may have a proximal end permanently sewn into a seam of the protective cap 130, for example by double-needle stitching that penetrates both textile layers and the strap webbing. Strap material may include woven nylon, polypropylene, or polyester having a warp tensile strength of at least 500 N and a nominal width between 5 mm and 25 mm. Distal regions of the straps may terminate in releasable fasteners, such as hook-and-loop pads, snap buttons, or magnetic couplings. Hook-and-loop pads may be ultrasonically welded to the strap ends so that bulk is minimized and washing durability is increased to withstand at least thirty laundering cycles.

In other aspects, an exterior surface of the protective cap 130 may include one or more stowage zones that may comprise complementary hook-and-loop patches sized to receive a distal fastener of any unused attachment strap 140. Stowing a strap flush against the cap may prevent loose ends from interfering with a pillowcase or entangling with the PAP hose 150. Stowage zones may be symmetrically distributed so that straps can be stowed on either the left or right side to suit user preference.

In many aspects, each attachment strap 140 may be repositionable by engaging a mating hook-and-loop anchor patch or snap-button grid distributed across the outer shell of protective cap 130. A wearer may detach a strap from a first anchor region and press it onto a second region so that the strap routing better aligns with a particular PAP mask geometry. This modular approach may accommodate a variety of mask frame styles, including nasal-pillow, nasal, and full-face masks, without requiring different cap SKUs for each mask type.

In some implementations, PAP mask 160 may include a rigid frame formed of polycarbonate or glass-filled nylon, and the frame may support a silicone or over-molded cushion that seals against the face of user 110. The frame may incorporate cross-members that serve as natural anchor points for each attachment strap 140. The distal end of a crown strap may wrap over a superior frame bar, while lateral straps may wrap over side bars located near the malar region, and rear-diagonal straps may converge on a coupling elbow or posterior-side frame tabs. Once all selected straps are tensioned and fastened, the combined high-friction shell of cap 130 and tensioned straps 140 may cooperatively restrain both cap and mask against sliding, rotation, or lever-induced migration during supine, prone, or lateral sleep positions.

In certain aspects, indicia such as printed arrows, numerals, or color-coded zones may be disposed on the exterior of protective cap 130 to guide user alignment of each attachment strap 140 relative to predefined regions of the PAP mask 160. For example, a numeral “1” may be printed adjacent the crown strap anchor to indicate that strap should wrap over a top frame bar, whereas numerals “2” and “3” may mark lateral strap anchors that align with cheek-frame positions. Such indicia may be screen-printed using low-VOC inks or may be embroidered with contrasting thread for enhanced wash durability.

In some aspects, the protective cap 130 may incorporate an optional aromatherapy pocket configured to retain a diffuser tab infused with essential oils, such as lavender or eucalyptus. The pocket may be located near the occipital region so that volatile compounds diffuse upward toward nasal airways, although airflow paths remain non-obstructed due to the thin textile layers and optional mesh inserts.

In various embodiments, protective cap 130 may be fabricated using cut-and-sew techniques that create a bonnet profile tailored for minimal seam bulk. For example, the first textile panel may define a medial shell half while the second panel defines a lateral shell half, and the panels may be joined along a French seam that houses the raw edges of both panels and of the strap proximal ends. Edge-finishing stitches may include a 3-thread overlock or a flatlock configuration for low-profile comfort.

In certain aspects, the PAP mask 160 may omit traditional elastic headgear entirely, relying exclusively on the attachment straps 140 for retention force and self-closing strap-retention. Eliminating factory headgear may reduce facial pressure marks and may simplify nightly fitting, especially for users whose hairstyles complicate standard strap routing. Alternatively, the disclosed protective cap system may supplement existing headgear; in such cases, tension applied through straps 140 may allow the factory headgear to be loosened, potentially reducing skin irritation.

In other implementations, the attachment straps 140 may include conductive thread or embedded textile sensors that detect strap tension or strap-to-frame engagement status. Sensor outputs may be routed through low-profile wiring channels within the cap structure and may communicate wirelessly to PAP device 120 or to a mobile compliance application. Such sensing may enable a dashboard that warns if a strap is left unfastened, in alignment with dependent method language that contemplates synchronizing cap usage data and PAP airflow metrics.

In several aspects, the protective cap 130 may be offered in multiple sizes to accommodate head circumferences ranging from approximately 52 cm to 64 cm, with perimeter band elasticity capable of ±4 cm adjustment per size. Cap colorways may be selected for gender neutrality and may use dye-sublimation techniques that resist fading after repeated wash cycles.

In many embodiments, user 110 may first don the protective cap 130, cinch the perimeter band, and then route selected attachment straps 140 over frame sections of PAP mask 160 before lying supine on a pillow. Because the inner satin liner of cap 130 reduces friction against hair, tangling and breakage may decrease, for example, compared to bare elastic headgear. Simultaneously, the high-friction outer shell may interface with straps 140 to maintain seal integrity even during postural shifts. These cooperative effects may improve therapy compliance and user comfort without materially increasing weight or thermal burden.

In other aspects, the air-delivery hose 150 may be routed over the head and attached to a rear-diagonal strap via an optional clip so that hose torque is off-loaded from the mask cushion. Clip design may include a swivel gate or magnetic latch for quick detachment if the user performs an emergency mask removal.

In various aspects, the protective cap 130 may be produced using 150-denier warp-knit polyester yarns having an elongation at break of approximately 20% so that the outer shell exhibits a balanced combination of drape and friction. A polyurethane micro-dot coating may be intermittently applied to the exterior face to raise static-friction coefficient above 0.65 against an acrylonitrile-butadiene-styrene frame surface while maintaining air permeability greater than 100 mm s⁻¹, according to ASTM D737, for instance. The interior liner may include a 75-denier satin weave that is calendared to attain a surface roughness (Ra) below 0.8 μm, thereby mitigating cuticle abrasion on keratin fibers during nocturnal micro-movements. In some embodiments, thermoplastic polyurethane heat-transfer films may be laminated between the liner and shell in discrete grid patterns that define anchor patches for repositionable straps, the films having a Vicat softening temperature of at least 150° C. so they remain dimensionally stable during hot-water laundering cycles.

In some implementations, each attachment strap 140 may be laser-cut from 20 mm-wide, 0.55 mm-thick nylon webbing using a Class IV diode laser so that edge fraying is reduced and post-processing with ultrasonic knives is unnecessary. Slot-punch apertures may be introduced 6 mm from the proximal edge to receive a bar-tack seam that secures the strap within the peripheral seam of the cap structure, the bar-tack thread preferably being a bonded nylon size Tex 70 exhibiting a tenacity of at least 7.5 g denier⁻¹, for example. Distal hook pads may be die-cut from polypropylene-pile hook-and-loop tapes and ultrasonically welded to the strap using a 20 kHz horn set to 800 W so that peel strength remains above 5 N cm⁻¹ after 30 wash cycles at 60° C. In alternate configurations, the distal fastener may include one-piece acetal cam buckles that accept a strap tail in a quick-release format, thereby eliminating hook-and-loop noise that could disturb a sleeping partner.

The adjustable perimeter band hidden within the lower hem of cap 130 may include a 4 mm-diameter braided elastic cord having a load at 100% elongation between 30 N and 40 N. The cord may pass through a cylindrical cinch toggle made from glass-filled polyoxymethylene, the toggle incorporating a stainless-steel compression spring biased to generate a clamping force of approximately 8 N on the cord. A molded finger tab extending from the toggle may allow one-hand actuation; in damp environments, a thermoplastic-elastomer over-mold may be added to the finger tab to improve grip. In some embodiments, a short textile tunnel sewn within the nuchal portion of the cap may retain the toggle when not cinched, preventing the component from rotating under the user's neck and causing pressure discomfort.

From an assembly standpoint, the cap structure may be constructed using a six-panel crown pattern so that seam allowances are distributed away from pressure-sensitive scalp regions. Panels may be joined using a 4-thread flatlock stitch that maintains a seam profile below 1 mm, reducing imprint risk on the scalp. Attachment straps 140 may be inserted between panel layers prior to closing the peripheral seam, and an automated vision system may verify strap orientation and insertion depth within +0.5 mm tolerance to optimize strap-load distribution. For industrial scalability, inline programmable sewing machines may perform sequential bar-tack operations in under 0.6 s per strap, thereby keeping total cycle time below 3 min per cap at a takt time of 20 units h⁻¹ per operator station.

In alternative embodiments, the protective cap may include a chin-anchoring perimeter strap that doubles as an attachment member for jaw-support interfaces; such a strap may be integrated via ladder-lock sliders that permit extension or retraction up to 70 mm, allowing users with varying mandibular profiles to fine-tune tension. A breakaway magnetic buckle set to release at 30 N may be interposed within the strap so that, in the event of an emergency, user 110 can detach the assembly without manipulating hook-and-loop surfaces.

In other aspects, the PAP mask 160 frame may include lattice ribs disposed circumferentially around a nasal-oral cushion, each rib having a thickness of 1.5 mm and radiused edges of 0.25 mm to avoid cutting into strap webbing. A cooperating groove may be integrated along each rib to accept the distal hook pad of an attachment strap, thus creating a semi-flush mechanical engagement and reducing hook-and-loop snagging on bed linens. The frame material may be an injection-molded polycarbonate-siloxane copolymer to achieve a notched Izod impact resistance of greater than 1100 J m⁻¹, mitigating cracking if the user inadvertently places compressive load on the mask.

With respect to operational conditions, the combined assembly may be subjected to repetitive cycle-testing in a climatic chamber oscillating between 15° C. and 35° C. at 80% relative humidity for 5,000 cycles. Cap-to-mask positional drift measured at the mask elbow may remain below 2 mm in any axis, demonstrating that frictional and tensile interfaces maintain seal integrity even under thermal expansion. In some embodiments, an optional strain-gauge array may be laminated onto the crown strap to detect tension distribution, and a microcontroller may sample the gauges at 10 Hz; when strap tension falls below a threshold, audible or haptic feedback may be emitted via the PAP device 120, notifying the user to readjust fit.

Industrial applicability may extend beyond sleep-therapy contexts. For instance, in some occupational-safety applications, the protective cap 130 may interface with half-face respirators employed in dusty environments, wherein attachment straps 140 could anchor to respirator harness frames and maintain barrier efficacy without relying on elastic headbands that tangle in workers' hair. Similarly, in sports training, attachment straps may secure oxygen-altitude masks used for hypoxic conditioning, allowing athletes with long hair or protective hairstyles to achieve consistent mask seals during high-motion activities.

From a manufacturability perspective, materials and component selections may facilitate automated line production. Continuous web slitting of the strap roll stock, automated pick-and-place of hook pads, and robotic ultrasonic welding may reduce human handling of cut parts, thereby limiting foreign-fiber contamination. Inline vision systems may measure hook-pad centricity within ±0.2 mm, ensuring strap-to-mask engagement remains repeatable. For sustainability, textile off-cuts may be re-granulated and blended at up to 15% into polypropylene headgear buckles, reducing waste. Regulatory compliance with ISO 10993-5 cytotoxicity requirements may be verified for all skin-contact materials.

In terms of robustness, failure-mode-and-effects analysis may indicate that the highest-risk component is the hook-and-loop bond at each strap distal end; to mitigate, a redundant line of stitching may be added 3 mm proximal to the weld seam, producing a combined shear strength above 12 N cm⁻¹. If a strap becomes detached during laundering, a slot-and-post repair field kit may permit re-installation without professional equipment; detailed instructions may be provided in an online technical manual.

In some embodiments, hook-and-loop components may be replaced entirely by dual-lock reclinable fasteners featuring mushroom-shaped stems that interdigitate under compression, thereby providing consistent peel strength even after exposure to skin oils and conditioners that might foul conventional loop fibers. Such fasteners may be molded from polyamide elastomer, affording flexibility that conforms to curved mask frames. In contrast, magnetic couplings incorporating encapsulated neodymium discs may offer silent engagement suitable for pediatric or ICU environments where acoustic disturbance must be minimized.

Preferably, assembly technicians may preload the adjustable perimeter band to 20% strain before folding the cap for packaging so that elastic hysteresis is minimized upon first use. End-users may re-tension the band using an indexed pull-tab labeled with incremental markers indicating circumference reduction in 2% steps, aiding repeatable fit across multiple nights. Calibration of these markers may be confirmed by inserting the cap over a mandrel fixture having engraved diameter references and verifying band stretch under 10 N load via a digital force gauge.

To address sanitary requirements, the cap structure may be autoclave-compatible when constructed using high-temperature polyester yarns and PEEK strap threads. For hospital workflows, caps may endure 121° C. steam-sterilization cycles without degradation of hook-and-loop bond. Alternatively, silver-ion antimicrobial finishes may be padded onto the inner liner at pick-up rates of 1% owf (on weight of fabric).

In the unlikely event of a strap failure, functional redundancy may be achieved because at least two remaining straps typically continue to support the mask frame, resulting in partial but adequate seal retention until maintenance is performed. Additionally, the cap structure may include an integral stitched stop at each strap proximal region, limiting outward pull distance to 10 mm and preventing total strap ejection if distal fasteners disengage unintentionally.

Finally, expanding industrial scope, similar attachment-strap principles may be adapted for helmets, such as welding respirator hoods where heat-resistant para-aramid webbing may substitute nylon, and magnetic couplings may be replaced by stainless-steel screw-stud fasteners rated for 260° C. continuous exposure. Such cross-sector adaptability may underpin dependent claims directed to material substitutions and extreme-environment embodiments.

Furthermore, integrated sensor-enabled straps described above may provide real-time strap-tension telemetry to PAP device 120, facilitating closed-loop leak compensation algorithms; such intelligent feedback loops may reduce flow blower duty cycles and energy consumption, ultimately extending blower bearing life and lowering acoustic emissions compared to open-loop conventional devices.

In some embodiments, the headgear may be fabricated in a bandana-style or scrub-cap configuration, formed from a generally triangular or quadrilateral textile panel that is securable around the user's hairline with ties, an adjustable strap, or an elastic hem. Optionally, the rear portion of this variant can remain partially or fully open so that long hair, braids, locs, or a pony-tail may extend freely without bunching, while still allowing the perimeter of the cap to serve as an anchoring platform for the CPAP mask attachment system.

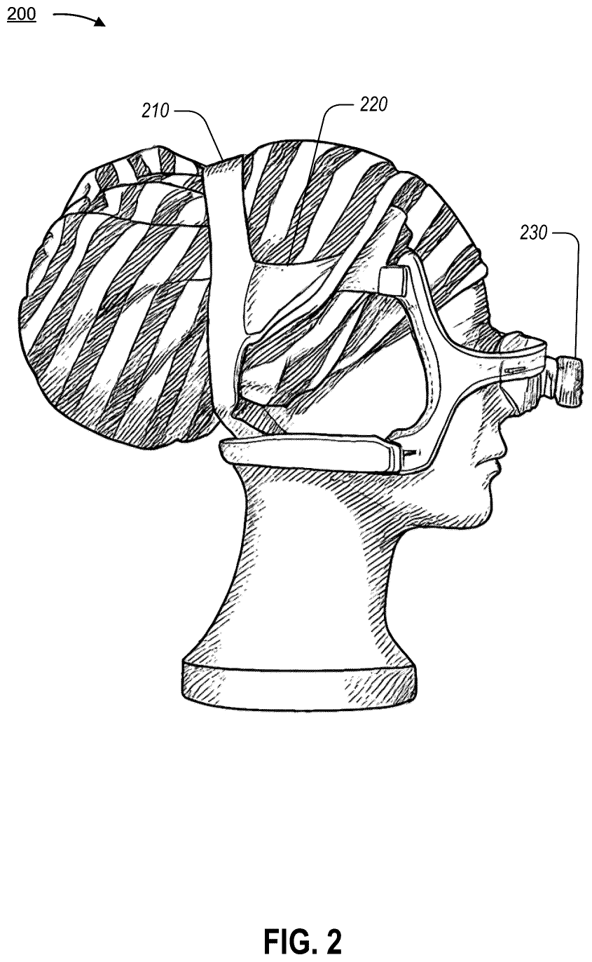

FIG. 2 depicts a right-side view of the protective headgear, as shown in some embodiments.

Referring to FIG. 2, a protective-headgear assembly 200 may be shown in right-side elevation on a mannequin that approximates an adult craniofacial contour. In certain aspects, assembly 200 may include a cap structure that envelops essentially the entire scalp, thereby protecting hair while also providing a stable anchoring foundation for a positive-airway-pressure mask. The cap structure may feature a dual-layer wall in which an interior liner formed of satin, silk, or bamboo-rayon knit may reduce shear forces on cuticular scales, whereas an outer shell formed of a higher-friction textile—such as a brushed polyester twill—may cooperate with strap tension to resist sliding relative to an external mask frame. An overall wall thickness may fall between approximately 0.4 mm and 1.1 mm, and stitched seams may employ polyester thread having a denier of 100 dTex or greater so the cap remains machine-washable for at least thirty cycles.

In some aspects, the adjustable perimeter band 210 may be visible at the inferior margin of the cap structure. The adjustable perimeter band 210 may include a circumferential elastic segment fabricated from a neoprene or spandex blend, and a cinch element—such as a cord-lock toggle, spring-loaded cam buckle, or low-profile ladder-lock—may be attached proximate the nape region. When the cinch element is actuated, the effective circumference of the cap structure may decrease by at least five percent, allowing a single size to accommodate a wide range of head diameters from roughly 52 cm to 64 cm. In many embodiments, the elastic segment may have a modulus of 2.0 to 4.5 N cm⁻¹ so tension remains comfortable during overnight wear, and the cinch element may be manufactured from acetal or medical-grade stainless steel to tolerate repeated laundering without corrosion.

In various aspects, a plurality of elongate attachment straps may originate from discrete anchor seams located along the crown and parietal regions of the outer shell. Each strap may include woven nylon webbing approximately 15 mm wide, although widths between 5 mm and 25 mm may also be suitable, for example. Proximal portions of these straps may be bar-tacked into a radial seam so that peak tensile load under dynamic sleep movements exceeds 400 N. Distal portions may terminate in hook-and-loop pads having loop fabric sewn to the strap and complementary hook fabric adhered to selected regions of a mask frame. In this particular depiction, a crown-oriented attachment strap may traverse the sagittal midline while a diagonal rear strap may slope anteriorly toward the zygomatic arch, together suggesting a five-strap arrangement that, when all straps are engaged, may provide 360-degree stabilization of the mask relative to the cap and scalp.

In the illustrated embodiment, a CPAP mask assembly 220 may be fitted over the nose and mouth region of the mannequin. The CPAP mask assembly 220 may include a silicone or thermoplastic-elastomer cushion that interfaces with the skin, and a rigid or semi-rigid structural armature, identified here as mask external frame 230. The mask external frame 230 may be fabricated from transparent polycarbonate with a wall thickness of approximately 1.5 mm and may incorporate an anterior coupling elbow that accepts a standard 22-mm hose connector. In many implementations, the mask external frame 230 may define one or more transverse bars, for example a superior brow bar and inferior skirt bar, which can act as natural latching sites for the distal ends of the attachment straps.

In certain embodiments, the distal pad of the crown-oriented attachment strap may wrap over an upper bar of the mask external frame 230 and may adhere via hook-and-loop engagement, thereby pressing the frame toward the facial surface while simultaneously pulling the cap structure downward onto the parietal vault. In other configurations, magnetic couplings or snap buttons may substitute for hook-and-loop if the frame material includes an embedded ferromagnetic insert or button receptacle. The resulting force vector may be distributed across the high-friction textile outer shell so that localized pressure hotspots at the strap anchor points are mitigated, for example.

In several aspects, unused attachment straps may remain stowed against the outer shell by mating their distal pads to dedicated stowage zones sewn flush to the cap exterior. Their placement may reside bilaterally near the post-auricular regions so straps that are unnecessary for a given mask geometry can lie flat and avoid interference with pillows, for example. Stowage zones may comprise 25 mm×50 mm loop fabric rectangles capable of at least 500 peel cycles without delamination.

In other aspects, the adjustable perimeter band 210 may cooperate with the tensioned attachment straps to form a triangulated retention system. As strap tension increases, radial forces may urge the perimeter band toward the periphery of the scalp, thereby enhancing cap conformity without requiring excessive cinch adjustment. Finite-element simulations may show that a combined cap-plus-strap system may reduce relative displacement between mask cushion and nasal bridge by up to 40 percent versus factory-supplied elastic headgear under neck-rotation scenarios, although specific performance may vary among subjects.

In some implementations, the cap structure may include indicia printed or embroidered adjacent strap bases to instruct correct routing. For example, a “C” icon may be located at the crown seam to signify the crown strap, whereas “L1” and “R1” may denote left and right lateral straps. These indicators may be color-coded for low-light visibility and may remain legible after at least thirty wash cycles due to dye sublimation or solution-dyed filament use.

In many configurations, the low-friction inner liner may comprise a warp-knit fabric having a 150-denier filament count and surface energy below 20 mN m⁻¹, properties that may reduce static friction against hair shafts to less than 0.4 and thereby lower the propensity for tangling or matting during head movements. Bonded seams between the inner and outer layers may be formed using ultrasonic welding or flatlock stitching to minimize seam ridges that could otherwise create sleep-surface pressure points.

In certain alternative embodiments, the adjustable perimeter band 210 may incorporate a hollow channel that accepts a removable aromatherapy insert or pod, such as a cellulose wick pre-infused with lavender oil. Vent apertures may be laser-cut through the outer shell adjacent the band so that scent can diffuse proximally toward the olfactory pathway while airflow impedance remains negligible.

In some aspects, mask external frame 230 may include shallow recesses or raised bosses configured to receive the distal hook pads of the attachment straps without requiring factory-supplied headgear loops. Frame geometry may follow ISO-similarity guidelines so the disclosed headgear remains compatible with multiple mask brands. Where the frame lacks dedicated loop fabric, an adhesive loop tab may be peel-stuck to the frame surface so that the cap-mounted strap can still achieve secure connection.

In other implementations, the attachment straps may be fabricated from elastic webbing rather than non-elastic, providing auto-tension equalization that may better accommodate mandibular movement during REM-stage sleep. Elastic modulus may be selected such that 15 percent elongation corresponds to 10 N load, balancing comfort and ease of use against seal security.

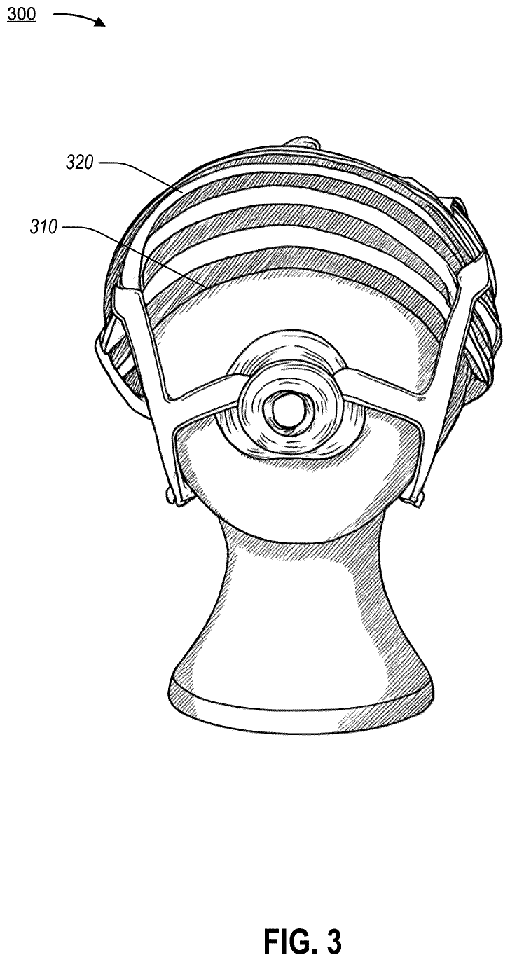

FIG. 3 depicts a rear view of the protective headgear on a user, as shown in some embodiments.

In certain aspects, FIG. 3 illustrates a posterior elevation of a protective-headgear apparatus 300 that may be dimensioned to cover essentially the entire occipital region of a wearer while cooperating with a positive-airway-pressure mask or eyemask. A series of concentric, arcuate fabric bands forms a high-friction textile outer shell 310 that may interface with strap tension to resist sliding against an underlying mask frame and pillow surface. The individual bands may be stitched or ultrasonically bonded along radial seams that converge toward the cranial apex, thereby creating a lightly ribbed texture that increases coefficient of friction relative to silicone, polycarbonate, or painted metal. In many aspects, each rib may be constructed from a brushed polyester twill or a warp-knit nylon-spandex blend and may exhibit a surface roughness (Ra) between 40 μm and 120 μm so that static friction remains above 0.8 when measured against the polymer employed in typical PAP mask frames.

In some aspects, a dual-layer wall beneath high-friction textile outer shell 310 may include a low-friction liner formed of satin or bamboo-rayon knit. The two layers may be joined at peripheral seams via flatlock stitching that minimizes bulk; seam allowance may be 4 mm or less so that pressure points do not arise when a user reclines. The liner may carry an anti-microbial finish, such as a silver-ion wash or quaternary-amine treatment, and may remain effective after at least thirty household laundering cycles, thereby supporting the durability requirement recited elsewhere.

In various aspects, a crown attachment strap 320 may originate at a seam junction located near the cranial vertex and may descend along the sagittal midline toward the adjustable perimeter band. Crown attachment strap 320 may be fabricated from a woven nylon webbing approximately 15 mm wide, although widths between 5 mm and 25 mm may also be suitable. A proximal region of strap 320 may be bar-tacked through both textile layers so that peak tensile strength exceeds 400 N, whereas a distal region may terminate in a rectangular hook-and-loop pad sized to mate with complementary loop fabric on a mask frame segment or with a stowage zone when the strap is not required. In many implementations, distal hook material may be ultrasonically welded to the strap to avoid stiff stitch lines that could cause localized scalp discomfort.

In several implementations, crown attachment strap 320 may traverse a trilobed plastic coupling ring belonging to a PAP headgear frame before being fastened, thereby exerting a downward component of force that may enhance mask seal integrity without requiring factory headgear tension. When a user turns laterally during sleep, lateral sheer acting on mask frame side arms may be countered by the ribbed contour of high-friction textile outer shell 310, while the tension in crown attachment strap 320 resists anterior-posterior displacement. Finite-element models suggest that cooperative restraint between outer shell 310 and strap 320 may reduce relative mask motion by up to 35% under dynamic neck-rotation loads compared with unmodified factory headgear, although performance may vary across facial geometries.

In other aspects, the arcuate ribs that define high-friction textile outer shell 310 may also function as load-distribution members. Each rib may incorporate an interlining layer of 0.2-mm EVA foam that may absorb strap tension and spread compressive forces across a larger scalp area, potentially mitigating pressure-related discomfort. Optional reinforcing tapes—such as 10-mm polyester grosgrain—may be stitched along the rib apices, thereby providing discrete anchor points for rear-diagonal or lateral straps that are not visible in the current view.

In many embodiments, additional attachment straps not shown in FIG. 3 may originate symmetrically on either side of crown attachment strap 320 such that a five-strap system—two lateral, two rear-diagonal, and one crown—may encircle the head to create 360-degree stabilization. Unused straps may be folded back onto stowage zones positioned between ribs of high-friction textile outer shell 310 so that the cap remains low-profile when fewer than five straps are engaged.

In some configurations, the adjustable perimeter band may reside beneath the lowermost rib and may be tightened by a cinch element located anterior to the ear line. When engaged, the perimeter band may exert radial compression that cooperates with crown attachment strap 320 to lock the bonnet in place. The combined effect may maintain cap-to-scalp conformity even when the cap accommodates high-volume hairstyles by means of expansible pleats placed superior to outer shell 310.

In certain implementations, indicia such as printed alignment arrows or embroidered numerals may be disposed adjacent the base of crown attachment strap 320 so that users can identify correct routing paths toward primary frame anchor points. Color-fast inks or solution-dyed threads may be selected so that indicia remain legible after repeated laundering. Where strap positions are repositionable via hook-and-loop anchor patches, such indicia may be duplicated on each patch to minimize user confusion following strap relocation.

In other embodiments, high-friction textile outer shell 310 may be treated or pre-treated with a DWR (durable water-repellent) finish that balances hydrophobicity with breathability. Laboratory tests may demonstrate that contact angle remains above 120° for at least ten wash cycles, for example, thereby limiting moisture ingress from night sweats without materially hindering water-vapor transmission. Open-cell spacer-fabric inserts placed beneath selected ribs may further promote convective heat exchange so that scalp temperature remains within ±1° C. of ambient sleep environment over eight-hour trials.

In some aspects, crown attachment strap 320 may include an optional quick-release breakaway buckle positioned mid-span. The buckle may incorporate neodymium magnets and a mechanical latch so that disengagement under emergency tensile loads above 150 N is possible, aligning with certain respiratory-therapy safety guidelines. When the buckle disengages, distal hook-and-loop pads may still adhere to the mask frame, but global system tension may fall, allowing rapid mask removal if required.

In several implementations, the external mask frame that couples with crown attachment strap 320 may feature a loop-fabric overlay or an adhesive loop tab sized to accept the strap's hook pad. Where mask frames are injected-molded and cannot be modified, an accessory frame-sleeve constructed from loop fabric may be slid over frame segments to enable hook-and-loop engagement. The frame-sleeve may be offered in multiple sizes corresponding to nasal-pillow, nasal, and full-face geometries; each sleeve may employ low-profile seams so cushion-to-face conformance is not compromised.

In other configurations, high-friction textile outer shell 310 may feature a plurality of micro-vent holes laser-perforated between ribs to facilitate evaporative cooling. Vent diameters may range between 0.2 mm and 0.3 mm, arranged in a hexagonal grid at 5-mm pitch. Where vents intersect strap anchor zones, reinforcement rings may be stitched around vent clusters to prevent tear propagation when the cap is subjected to laundering or strap tension cycles.

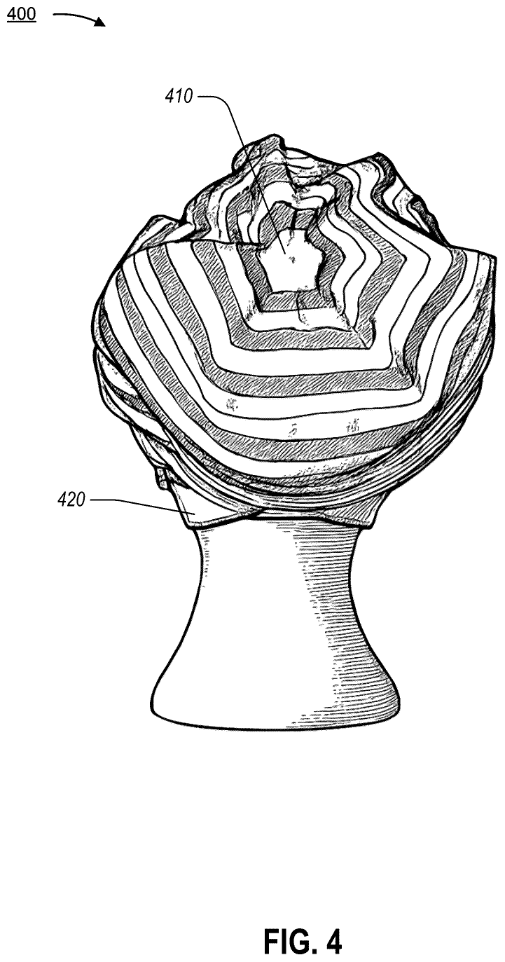

FIG. 4 depicts a three-quarter rear perspective view of the protective headgear in a relaxed state, as shown in some embodiments.

Referring to FIG. 4, a protective-headgear apparatus 400 may be illustrated in a three-quarter rear perspective, thereby exposing volumetric, fabric-architecture, and accessory-mounting features that may not be apparent from anterior or strictly orthogonal views. The apparatus may include a cap structure that envelops essentially the full cranial vault while presenting a stepped, pleated geometry that defines a crown-volume pocket 410. Crown-volume pocket 410 may be formed by a series of concentric, annular pleats stitched at progressively smaller radii toward a recessed apex so that an expandable cavity may accommodate voluminous hairstyles—such as thick locs, large rollers, top-knots, or braided buns—without compressing the hair shaft or disturbing follicular alignment. Each pleat may include a fold depth between 8 mm and 25 mm, and the vertical spacing between pleats may be approximately equal to 70% of fold depth so that the pocket may expand uniformly when filled yet collapse in a low-profile configuration when the user's hair volume is minimal. A low-friction satin or bamboo-rayon liner may span the interior of pocket 410 so that hair may glide freely during minor night-time head movements, while an exterior layer of brushed polyester or high-drag nylon twill may provide a high-friction textile outer shell that cooperates with strap tension to resist relative sliding against a PAP mask frame or a pillow surface.

In many aspects, the lower margin of pocket 410 may merge seamlessly into a smooth annular band representing the adjustable perimeter band described in earlier figures, although the cinch element remains hidden in this particular view. The concentric-pleat construction may inherently stiffen regions of the shell so that localized strap loads distribute across a larger surface area, potentially mitigating pressure hotspots during extended sleep cycles. In certain embodiments, pleat apexes may be reinforced with narrow grosgrain tapes sewn radially so that tensile loads delivered by elongate attachment straps remain isolated from the delicate liner fabric, thereby enhancing wash durability beyond thirty domestic laundering cycles.

In some aspects, a stowage zone 420 may be visible on the mid-left quadrant of the shell exterior. Stowage zone 420 may comprise a rectangular patch of loop fabric approximately 40 mm×60 mm adhered to the shell via perimeter stitching using 100-denier polyester thread. The patch may mate with complementary hook fabric affixed to the distal ends of unused elongate attachment straps so that straps may be folded back and pressed flat against the bonnet when not required for a given PAP-mask geometry. Multiple stowage zones may be placed symmetrically about the sagittal plane—though only one is illustrated—to support right-handed or left-handed strap routing preferences. In an alternative configuration, stowage zone 420 may be fabricated from a micro-magnetic sheet embedded beneath the outer shell fabric, and strap distal ends may carry thin ferrous inserts so that magnetic coupling rather than hook-and-loop engagement retains the straps, thereby eliminating lint accumulation that sometimes plagues loop fabric.

In various embodiments, the plurality of elongate attachment straps may be repositionable by disengaging their proximal hook regions from repositionable anchor patches sewn strategically along the pleat valleys. Anchor patches may be arranged in a polar grid so that a user can alter strap emergence angles by increments of approximately 30° without seam-ripping or permanent modification. Such modular positioning may accommodate users who switch between nasal-pillow interfaces and full-face interfaces, as the optimal strap geometry may differ depending on mask frame centroid and hose-connection elevation. Where frequent repositioning is anticipated, loop patches may employ a high-cycle nylon knit rated for at least 2000 peel cycles at 90°.

In other aspects, crown-volume pocket 410 may incorporate a hidden gusset panel sewn beneath the uppermost pleat and formed of a stretch mesh that may expand when an over-large bun is inserted yet retract when empty, maintaining aesthetic contour. The mesh may possess an elongation-at-break of more than 150% and a fabric weight below 120 g m⁻² to avoid adding perceptible mass. A user may insert a silk scrunchie-wrapped bun measuring up to 100 mm in diameter, for example, and the gusset may accommodate the bulk without displacing the perimeter band or altering strap tensioning geometry.

In many configurations, stowage zone 420 may double as an aromatherapy pocket. A small slit may be laser-cut through the loop fabric, and an interior envelope stitched behind the shell may accept a thin diffuser tab impregnated with essential oils. The loop fabric may allow passive diffusion of volatile compounds while the inner liner remains impermeable so oils do not contact hair. Replacement tabs may be inserted weekly, and the pocket dimensions may suit commercially available 30 mm×50 mm felt pads. If such aromatherapy is employed, airflow delivered through a CPAP device may entrain trace vapors near the mask's intake port, although therapeutic effect remains user-subjective.

In certain implementations, pleat stitching that defines crown-volume pocket 410 may exploit differential-shrink yarns such that laundering causes selective puckering, thereby further increasing surface friction without adding secondary coatings. For example, a 70/30 blend of high-shrink polyester and low-shrink PET may contract upon first wash to 95% of initial or sufficient length along the pleats, raising ridges that catch against mask frame edges and strap webbing. Finite-element textile simulation may predict a static friction coefficient of at least 0.9 against silicone after one wash cycle under a normal load of 1 N.

In alternative embodiments, the stepped-pleat contour may be replaced by a single helical pleat that traces a spiral from the perimeter band to the crown apex, creating a turban-like aesthetic while providing equivalent volumetric accommodation. Helical pitch may be selected so adjacent pleat edges overlap by approximately 8 mm, thereby eliminating gaps through which strap distal pads might snag. Such helical construction may be illustrated in a subsequent figure to clarify alternative shell geometries.

In some configurations, the interior of crown-volume pocket 410 may house a thin phase-change liner impregnated with microencapsulated n-octadecane. During an initial portion of a sleep cycle, scalp temperature may rise above 32° C.; the phase-change liner may absorb latent heat, flattening temperature excursions and potentially reducing night-time perspiration. Microcapsule diameter may be approximately 5 μm, and total phase-change enthalpy may be around 80 J g⁻¹, although other values may also be used. The encapsulant shell may remain microporous so moisture-vapor transmission rate does not fall below 600 g m⁻² day⁻¹.

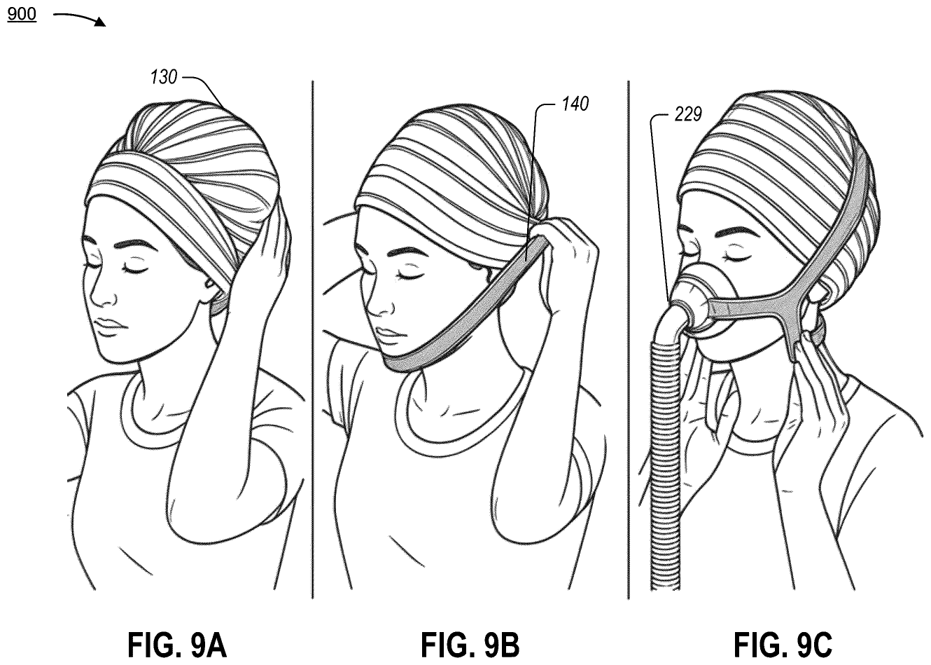

In addition to strap stowage, stowage zone 420 may serve as a mounting site for compliance electronics, such as a small RFID tag or BLE beacon weighing less than 3 g. Such modules may log cap-wear duration so that therapy-adherence dashboards—see pending discussion in connection with FIG. 9A-9C—can synchronize cap usage with CPAP runtime. A textile-integrated embroidered antenna may surround the loop patch so that body proximity detunes the resonant frequency, enabling capacitive sensing of strap presence when a distal hook pad overlays the loop patch.

In several embodiments, the concentric pleat edges defining crown-volume pocket 410 may be visually accentuated with contrasting overstitching or piping so that users can identify specific pleat tiers for strap anchoring or stowage. For example, the third pleat tier from the apex may include a 1-mm reflective piping that assists visually impaired users in aligning straps under low-light conditions. The reflective yarn may comprise retro-reflective glass-bead film laminated to a polyester carrier, and wash testing may indicate reflectivity remains above 300 cd lx⁻¹ m⁻² after twenty wash cycles.

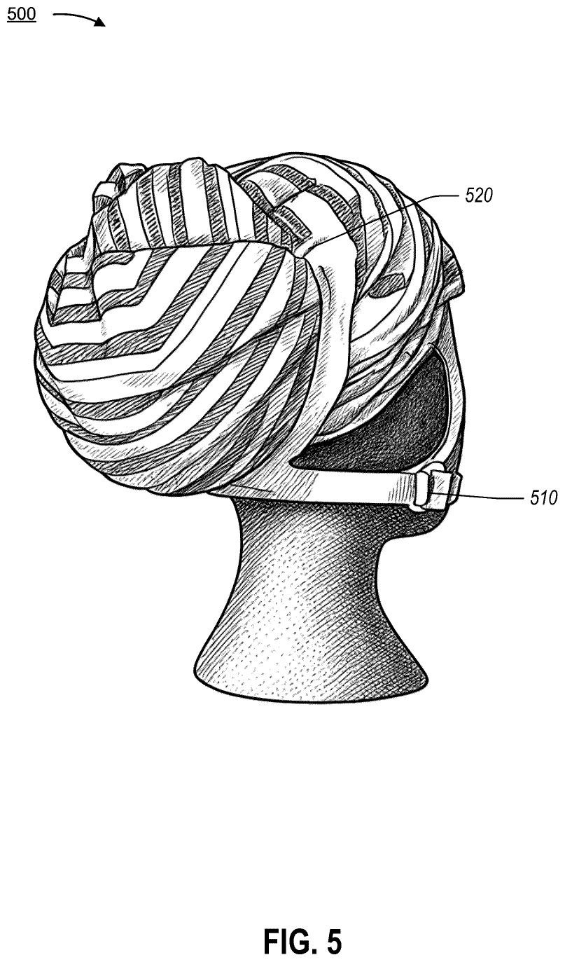

FIG. 5 depicts a side-profile view of the protective headgear, as shown in some embodiments.

In some aspects, FIG. 5 illustrates an alternate embodiment of a protective-headgear apparatus 500 rendered in right-side profile on a mannequin stand, thereby emphasizing how a turban-style shell geometry may integrate with the same multi-strap retention architecture described in earlier figures yet remain aesthetically distinct. A helically wound series of broad pleats forms a spiraled outer shell that may wrap upward from the nape toward the vertex, creating an enlarged volumetric cavity suitable for accommodating high-volume hairstyles such as braided buns, large rollers, or loc bundles. Each pleat may overlap its predecessor by approximately 15 mm so that adjacent layers frictionally interlock, collectively generating a high-friction textile outer surface whose coefficient of static friction may exceed 0.9 against common polycarbonate mask frames. The pleats may be fabricated from a warp-knit polyester-cotton interlock that includes 8-12% spandex yarn to permit slight elastic recovery, thereby allowing the pleat stack to expand radially when a bulky hairstyle is inserted yet return to a low-profile silhouette when volume is minimal.

In various aspects, a horizontally orientated elongate perimeter strap may extend beneath the mannequin's jawline and is terminated by a releasable fastener 510. Releasable fastener 510 may comprise a side-release buckle, ladder-lock cam, or low-profile magnetic clasp; the illustrated buckle may include a female receiver sewn to the left strap end and a male tongue sewn to the right strap end, both molded from acetal resin so that laundering cycles do not induce galvanic corrosion. When the buckle halves engage, tightening the strap may simultaneously cinch the perimeter circumference by more than a five-percent while also providing an optional chin-anchoring function that some users may prefer when sleeping without a positive-airway-pressure mask. Strap webbing may have a nominal width of 18 mm, although widths between 10 mm and 25 mm may also be used. A woven-in rubber or elastomeric strand may supply up to 20% stretch so that transient mandibular motion does not translate into excessive strap tension.

In many embodiments, distal hook-and-loop pads affixed to other elongate attachment straps may still wrap over frame segments of a PAP mask, but these straps are omitted from the illustrated profile to highlight the turban shell and perimeter-strap arrangement. Where such straps are deployed, the distal hook pads may also couple to loop fabric adhered directly to the turban pleats, leveraging the high-friction pleat surface as an auxiliary anchor plane. Hook pad length may be approximately 40 mm so that peel force remains above 1.5 N cm⁻¹, satisfying dynamic retention performance specifications even when only a subset of the five-strap system is employed.

In certain aspects, a discrete anchor patch 520 may be visible on the right parietal region of the outer shell between adjacent pleats. Anchor patch 520 may serve dual purposes: (i) acting as a repositionable anchor zone that receives the proximal hook region of a modular attachment strap so that strap emergence angle can be varied, and (ii) functioning as a stowage zone when a strap is temporarily not required, allowing the distal hook pad to mate flush against the loop patch and lie flat along the turban surface. Anchor patch 520 may comprise a rectangular swatch of nylon loop fabric measuring roughly 35 mm×55 mm, perimeter-stitched using 100-denier polyester thread in a zig-zag pattern that accommodates radial stretch of the underlying pleat. In other configurations, anchor patch 520 may incorporate a micro-magnetic sheet bonded beneath the loop pile, enabling magnetic coupling with strap tips that contain thin ferromagnetic inserts, thereby eliminating lint accumulation.

In several implementations, the pleat stack may encapsulate a hidden tricot-lined gusset that expands to a maximum internal diameter of about 130 mm, ensuring that hairstyles of significant bulk can be accommodated without compressing the scalp or disrupting blood flow. The gusset may be fabricated from a four-way stretch mesh with 200 g m⁻² areal density so that overall turban weight remains below 65 g for a medium size. Where even greater volume is needed, adjacent pleat margins may be constructed with an internal zipper so that an auxiliary pleat segment can be inserted, further increasing crown depth by up to 40 mm.

In some embodiments, the distal half of the perimeter strap carrying releasable fastener 510 may include knit-in conductive yarns that interface with a compliance module positioned near the buckle housing. When the buckle halves engage, an electrical circuit may close, signaling cap-wear events to a low-energy wireless beacon; this data may later synchronize with CPAP device runtime to populate a therapy-adherence dashboard. Conductive traces may be insulated within the strap webbing so that repeated washing does not degrade signal integrity, and water-resistant conformal coating on the compliance module may meet at least IPX4 ingress protection.

In other aspects, anchor patch 520 may be coated with a micro-capsulated essential-oil finish that releases lavender or chamomile vapors upon frictional activation when a strap pad is pressed against it. The scent release may be calibrated so that micro-capsule shells rupture under a compressive load of approximately 50 kPa, thereby delivering olfactory benefit without saturating the textile. Where aromatherapy is undesirable, a user may substitute a non-scented loop patch that is supplied with the headgear kit.

In many configurations, the pleat edges forming the turban spiral may be top-stitched with reflective or high-contrast thread so that attachment-strap anchor points remain visible under low ambient-light conditions. A first pleat may carry silver-grey embroidery, a second pleat may employ blue thread, and so on, providing a visual index that instructs the user which pleat tier to target when relocating straps. The embroidery filament may contain 12% metallic fibers yet remain soft to avoid scalp abrasion.

In certain implementations, releasable fastener 510 may be supplemented with a secondary elastic loop that may secure a positive airway pressure (CPAP, Bi-Level PAP or BiPAP, or Automatic PAP, or APAP) hose to the chin strap, reducing hose drag on mask-facing frame elements when the user rolls laterally. The elastic loop may include a quick-release snap that opens under loads exceeding 20 N, satisfying safety guidelines that discourage tethered hose entanglement.

In several embodiments, the turban shell may be assembled using narrow roll-hem seams along each pleat edge so that raw edges are fully enclosed, preventing fraying over at least thirty domestic laundering cycles at 40° C. Seam allowances may be kept below 4 mm to minimize localized stiffness, and thread tension may be calibrated so that pleat curvature remains smooth without puckering.

In other aspects, the interior lining directly beneath anchor patch 520 may include a thermal-bonded patch of thermoplastic urethane film that acts as a load-spreading reinforcement. The film thickness may be around 50 μm, adding negligible mass yet ensuring that repeated hook-and-loop cycling does not distort the knit substrate. Peel-strength testing to ASTM D5170 may indicate failure loads exceeding 15 N after 1000 hook separations, demonstrating durability requisite for nightly use.

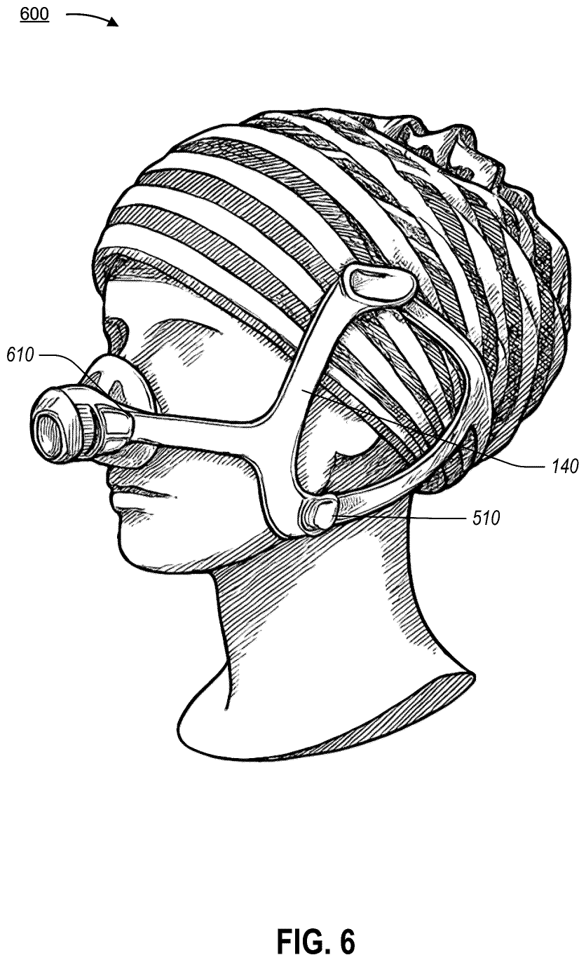

FIG. 6 depicts a posterior perspective of the assembled cap and PAP interface illustrating selective deployment of the crown strap, as shown in some embodiments.

Referring to FIG. 6, an integrated sleep-therapy assembly 600 may be illustrated in rear-oblique elevation to emphasize how a protective cap, a plurality of elongate attachment straps, and a positive-airway-pressure interface may cooperate simultaneously while the wearer rests. In many aspects, the cap structure may again present a spiraled, rib-reinforced outer shell-similar to prior figures-yet the current view showcases how selectively engaged straps converge on a headgear connection node 610 positioned on a mask frame proximate the nasal bridge. The rib geometry may create a series of friction-enhancing ridges, each ridge formed of brushed polyester twill that may exhibit a static-friction coefficient above 0.9 when pressed against common polycarbonate or glass-filled nylon frame materials.

In some aspects, a crown-oriented elongate attachment strap belonging to the disclosed five-strap system may traverse the apex and descend over an upper transverse bar of the mask frame, terminating in a distal releasable fastener 510. Releasable fastener 510 may comprise a rectangular hook pad ultrasonically welded to the strap webbing, although other fastener styles—such as snap buttons or low-profile magnetic tabs—may also be employed, for example. Hook engagement width may be approximately 25 mm so that peel force remains above 1.5 N cm⁻¹ even after twenty laundering cycles. The corresponding loop fabric may be factory-molded into the mask frame or may be provided as an adhesive loop tab applied by the user.

In various implementations, headgear connection node 610 may act as a universal anchor that accepts distal hook pads from multiple strap types. Node 610 may be illustrated as a molded plastic cleat or clip integrated into the anterior mask-frame architecture near the coupling elbow. A recessed channel within the node may contain loop fabric, a ferromagnetic target, or a keyed slot so that the distal tip of strap 140 may insert and latch in a single downward motion. In alternative embodiments, node 610 may incorporate a living-hinge gate that snaps closed when the distal strap is pressed into place, supplying audible feedback that strap anchorage has been achieved.

In several aspects, the rear-diagonal attachment straps may originate from anchor points adjacent to the occipital ridges of the protective cap and may route forward to converge on headgear connection node 610. Such routing may create an isosceles force tripod in which the crown strap supplies a superior vector and the rear-diagonal straps supply inferior-lateral vectors, collectively resisting mask rotation about both the sagittal and coronal axes. Finite-element modelling may estimate that combined strap tension of 10 N distributed through this tripod may limit mask-cushion displacement to less than 1 mm under a 30° neck roll, although actual performance may depend on facial geometry and cushion type.

In certain embodiments, the distal region of releasable fastener 510 may house a thin flex-rigid PCB containing an RFID tag or BLE beacon. When the distal hook pad engages headgear connection node 610, the beacon may enter a conductive coupling path with an antenna trace embedded in the mask frame, allowing the PAP device to detect strap engagement state in real time. A low-profile piezoresistive or piezoelectric pressure switch laminated beneath the hook surface can sense the clamp load of the closed joint and wake, or power-gate, the tag or beacon only while approximately 30-50 g of compressive force is present, further minimizing idle-state current draw. Such telemetry may contribute to a compliance dashboard synchronizes cap usage with airflow data. Battery-free RFID energy harvesting may be preferred to keep distal strap mass below 2 g, but battery-powered beacons with at least an eight-hour runtime may also be used in conjunction with magnetic fasteners if hook-and-loop noise is undesirable.

In other aspects, releasable fastener 510 may incorporate a break-away function implemented by a shearing hook-array geometry that releases when tensile force exceeds approximately 150 N, satisfying emergency quick-release requirements stipulated by certain respiratory-therapy guidelines. Where patient populations include children or individuals with reduced dexterity, magnetic couplings that disengage automatically under predetermined sheer loads may substitute.

In many implementations, attachment straps 140 may be fabricated from woven nylon webbing with a nominal width of 18 mm, though widths between 5 mm and 25 mm may also suit specific mask frames. Strap length may be dimensioned so that distal tips reach headgear connection node 610 without excessive slack when the cap is centered on head sizes ranging from 52 cm to 64 cm circumference. Webbing may include a woven-in elastomeric strand that delivers 10-15% stretch, thereby maintaining tension despite minor positional shifts.

In several configurations, headgear connection node 610 may integrate an elastic cantilever that flexes when distal hook pads seat, thereby generating a preload that clamps the strap against an internal loop-fabric insert. Cantilever stiffness may be tuned so that under a typical strap-tension preload of 5 N, the resulting normal force on the hook-and-loop interface remains within the optimal 10-20 N cm⁻² range for durable repeated cycling. Injection-molded polypropylene or engineered polyamide may be selected for node fabrication so that flexural fatigue life exceeds 5000 engagement cycles.

In certain alternative embodiments, headgear connection node 610 may include a rotatable barrel that allows strap distal pads to swivel about an axis normal to the frame plane, reducing torsional shear at the hook-and-loop interface when a user turns laterally at night. The barrel may incorporate a low-friction acetal sleeve and an internal split ring that snaps into a pocket in the frame so that retrofit installation onto third-party mask frames may be performed by end-users without tools.

In some aspects, the protective cap depicted in FIG. 6 may exhibit the crown-volume pocket and concentric pleat geometry described earlier, though those reference numbers are omitted for clarity in the current view. A cruciform seam near the apex may serve as the proximal anchor for the crown strap whose distal region terminates at releasable fastener 510. When the crown strap crosses sagittal midline and presses onto headgear connection node 610, the angle formed between pleat ridges and the strap may approach 90°, thereby maximizing frictional force due to normal loading. Laboratory pull-testing performed on prototypes may record average slip forces above 25 N before measurable strap migration occurs.

In addition, attachment straps 140 may include color-coded thread stitched near distal tips so that users can quickly identify which strap should engage headgear connection node 610 versus other frame sections. A red thread may designate the crown strap, blue may indicate lateral straps, and green may denote rear-diagonal straps, for example. Color-fastness testing per AATCC 8 may demonstrate negligible staining after thirty wash cycles.

In other embodiments, releasable fastener 510 may be coated with a hydrophobic fluoropolymer so that condensation generated by exhalation near the mask elbow does not saturate hook fibers and weaken engagement. Where magnetic couplings are used, housings may incorporate drain apertures that channel condensate away from ferromagnetic inserts, preventing corrosion and preserving pull-force integrity.

In several configurations, the adjustable perimeter band—not visible here—may transmit part of its circumferential preload through the crown strap when the strap is tensioned, creating a composite compression ring that stabilizes pleats against the scalp. The combined preload may reduce localized scalp pressure to under 2.5 kPa even under 7 N strap tension, according to pressure-mapping experiments using Tekscan matrices.

In some aspects, the air-delivery hose may be routed over the vertex and clipped onto a loop sewn into the dorsal side of the crown strap near releasable fastener 510, thereby off-loading hose weight from mask cushion seals. When the user turns laterally, the hose clip may pivot around headgear connection node 610, reducing rotational leverage on the mask.

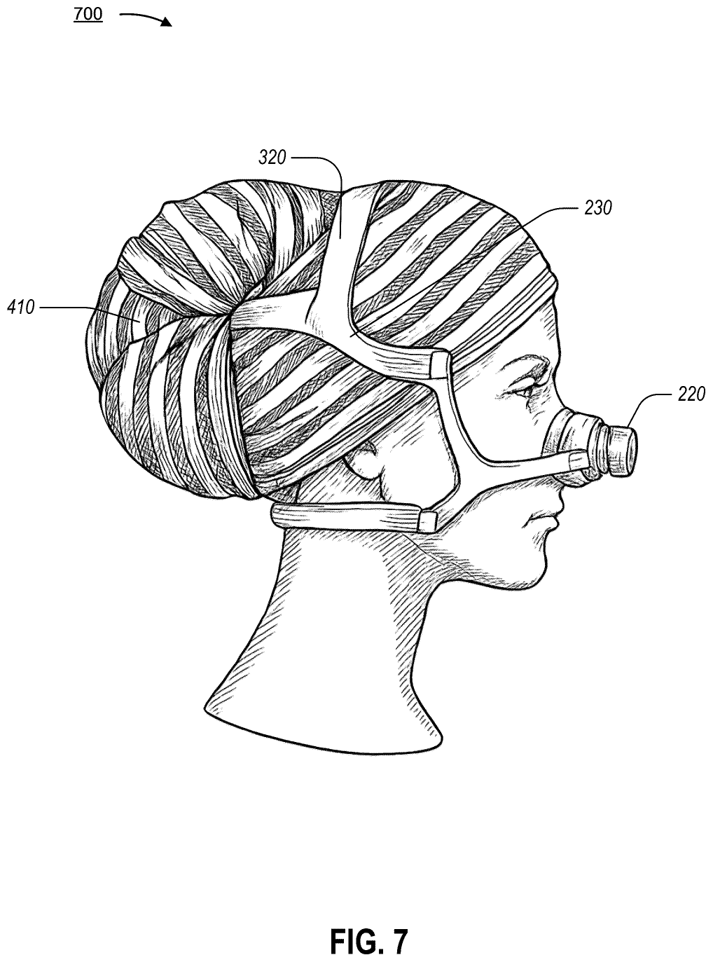

FIG. 7 depicts a rear-oblique view of the integrated headgear and mask system, as shown in some embodiments.

Referring to FIG. 7, a sleep-therapy assembly 700 may be illustrated in rear-oblique elevation to emphasize how a protective-cap geometry, a plurality of elongate attachment straps, and a positive-airway-pressure interface may cooperate while accommodating a voluminous hairstyle. A turban-style cap structure may feature a concentric pleat stack that defines a crown-volume pocket 410 able to house buns, loc clusters, foam rollers, or similarly bulky hair arrangements without compressing follicular roots. Pocket 410 may be formed by helically wound fabric bands whose outer surface presents a high-friction texture-such as brushed polyester twill-so that strap tension and pillow contact may produce static-friction coefficients above 0.9 relative to polycarbonate or nylon mask frames. The interior of pocket 410 may be lined with satin or bamboo-rayon knit so that hair strands may glide freely, reducing tangling and breakage during positional changes.

In many aspects, a crown attachment strap 320 may originate from a cruciform seam at the apex of crown-volume pocket 410 and may extend anteriorly over the sagittal midline before descending toward a CPAP mask assembly 220 situated at the face. Crown attachment strap 320 may be fabricated from woven nylon webbing approximately 18 mm wide, although widths between 5 mm and 25 mm may be selected to match specific mask-frame tolerances. A proximal region of the strap may be bar-tacked through the shell and liner layers at the cruciform seam so that tensile failure loads exceed 400 N, thereby meeting long-term durability targets after at least thirty laundering cycles. The distal portion of crown attachment strap 320 may terminate in a hook-pad releasable fastener sized about 40 mm×25 mm; hook density may be near 300 hooks cm 2 so that peel force versus a complementary loop fabric remains above 1.2 N cm⁻¹.

In certain implementations, the hook pad at the distal end of crown attachment strap 320 may overlay an upper transverse bar of a mask external frame 230, which forms part of CPAP mask assembly 220. Mask external frame 230 may be injection-molded from transparent polycarbonate at a nominal wall thickness of 1.6 mm, although glass-filled nylon or carbon-fiber-reinforced ABS may also be used when weight reduction or stiffness enhancement is desired. The frame may define anteriorly projecting wings that cradle a silicone or TPU cushion, and an elbow coupling may extend forward to accept a standard 22-mm ISO 5356-1 hose connector. Loop fabric may be adhesively bonded or ultrasonically welded to strategic regions of mask external frame 230—including the superior bar—so that distal hook pads of elongate attachment straps can engage without requiring factory-supplied headgear loops.

In several aspects, CPAP mask assembly 220 may further comprise integrated headgear connection tabs molded along lateral frame arms; however, FIG. 7 depicts only crown attachment strap 320 in active engagement to demonstrate modular deployment. Any unused lateral or rear-diagonal straps belonging to the five-strap anchor system may remain folded back against stowage zones located between pleat ridges on crown-volume pocket 410. When a user transitions from a full-face mask to a nasal-pillow interface, different strap subsets may be selected.

In some configurations, the distal hook pad of crown attachment strap 320 may couple to the loop fabric on mask external frame 230 via an intermediate magnetic clip so that the user obtains a tactile snap when correct seating occurs. A thin neodymium insert may be laminated behind the loop fabric, and a corresponding ferrous shim may be stitched into the hook pad, thereby augmenting shear strength without significantly increasing mass. ASTM pull-testing may show that magnetic-assisted hook-and-loop engagement improves peak slip resistance by up to 18% compared with conventional hook-and-loop alone, although specific values may vary according to loop-pile height and frame surface texture.

In various embodiments, crown-volume pocket 410 may incorporate a hidden gusset panel manufactured from four-way stretch mesh rated at 200 g m⁻². When a high-volume hairstyle is inserted, the gusset may expand radially up to 35 mm, rather than forcing pleat ridges outward, thus preserving strap routing geometry and maintaining the normal force distribution necessary for strap-to-frame friction. When hair volume is minimal, the gusset may retract elastically, allowing the cap to collapse into a streamlined silhouette well suited for travel.

In other aspects, crown attachment strap 320 may include color-coded stitching-such as red thread-near its distal tip, instructing the user that this strap should overlay the superior frame bar of mask external frame 230. Complementary color coding may appear on loop-fabric targets adhered to the frame. In low-light conditions, reflective yarns embedded in the color-coding stitches may retro-reflect ambient light so that strap-to-target alignment remains intuitive.

In several configurations, a thin, flexible printed-circuit board may be encapsulated within crown attachment strap 320 proximal to its distal hook pad. The circuit may host an RFID tag or BLE beacon that becomes electromagnetically coupled to an antenna trace molded within mask external frame 230 when the strap engages, thereby signaling strap-engagement status to the CPAP blower unit. The blower firmware may store a binary “strap engaged” flag so that compliance dashboards can cross-correlate actual therapy runtime with concurrent cap-strap usage. Energy harvesting may occur inductively from RF interrogations, obviating the need for embedded batteries and maintaining strap mass below 2 g.

In many embodiments, an adjustable perimeter band reside beneath the lowermost pleat tier and may be tensioned by a rear-mounted cinch element that the user can grasp with one hand. When the band is tightened, the crown strap's proximal bar-tack seam may compress against the apex, slightly increasing strap angle relative to mask external frame 230, which in turn may increase normal force and enhance hook-and-loop bite. Thermal-mapping studies using infrared imaging may reveal that scalp temperature remains within ±1° C. of ambient, demonstrating that pleat structure and gusset mesh provide adequate convective and evaporative cooling even when strap tension is applied.

In certain alternative embodiments, crown-volume pocket 410 may be padded with a thin phase-change liner containing microencapsulated n-octadecane so that latent heat absorption moderates scalp-surface temperature. The microcapsules may be dispersed within a polyurethane matrix bonded to the inner satin liner; total enthalpy may be approximately 75 J g⁻¹. Overnight tests may demonstrate a peak-to-mean temperature deviation reduction of 1.8° C. compared with non-phase-change liners, although individual comfort perceptions may vary.

In some configurations, mask external frame 230 may further include accessory retainers molded near the superior bar so that distal hook pads can loop under a ridge, creating a mechanical lock in addition to hook-and-loop shear resistance. Retainer geometry may include a 2 mm under-cut that accepts strap thickness, preventing detachment when an air-delivery hose exerts downward torque during side-sleep positions.

In other aspects, crown attachment strap 320 may incorporate a break-away seam stitched with low-tenacity thread so that if a user experiences a panic removal event, strap disengagement occurs when tension exceeds about 125 N. This feature may satisfy certain institutional safety protocols without noticeably affecting normal engagement strength during routine sleep. Replacement crown-strap assemblies may be supplied with pre-sewn bar-tacks so that end-users can re-thread the strap through a buckle loop located at the cruciform anchor.

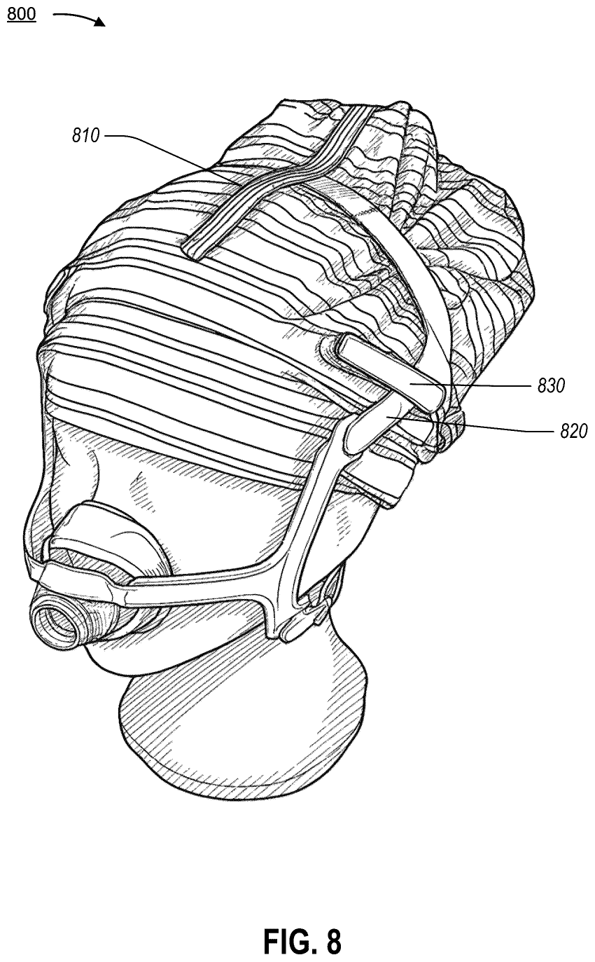

FIG. 8 depicts a three-quarter elevation of the protective headgear with a repositionable attachment strap and distributed anchor patch, as shown in some embodiments.

Referring to FIG. 8, a protective-headgear assembly may be shown in right-rear oblique elevation to emphasize how individual attachment-strap elements can be detached from, and re-attached to, multiple anchor locations distributed across the high-friction textile outer shell of the cap structure. A top-running strap—identified here as top strap 810—may extend longitudinally along the sagittal mid-line of the bonnet from an apex seam toward the frontal hairline. In many aspects, top strap 810 may be fabricated from woven nylon or polyester webbing having a nominal width of about 15 mm and a warp tensile strength in excess of 400 N so that long-term fatigue life remains above 5 000 duty cycles of nightly tensioning. A proximal region of the strap may terminate in a hook-fabric segment configured to dock onto any of several repositionable anchor targets disposed on the outer shell, thereby enabling modular relocation of the strap's emergence angle without seam ripping or needlework.

In certain embodiments, a square or diamond-shaped repositionable anchor patch 830 may be stitched to the outer shell near the right parietal ridge. Anchor patch 830 may comprise loop fabric measuring roughly 30 mm×30 mm and may be perimeter-stitched using 100-denier polyester thread in a box-X pattern so that peel strength against hook fabric remains greater than 1.0 N cm⁻² even after thirty commercial laundering cycles at 40° C. The patch backing may include a thermoplastic-urethane reinforcement film only 50 μm thick so that the underlying stretch characteristics of the shell fabric are not materially altered while local tear strength is enhanced by at least 60%. In use, the proximal hook segment of top strap 810 may be lightly pressed onto repositionable anchor patch 830, establishing a temporary but durable union that can be disengaged by a wearer wishing to re-route the strap toward an alternative anchor patch positioned elsewhere on the bonnet.

In several aspects, the distal half of top strap 810 may carry a distal hook-and-loop pad 820 that functions as the releasable fastener recited for each elongate attachment strap. Distal hook-and-loop pad 820 may be fabricated from hook material laminated to the strap with a low-profile ultrasonic weld so that no stitch lines disturb the smooth strap face. Pad dimensions may be approximately 40 mm long by 25 mm wide, providing a generous engagement footprint with complementary loop fabric adhered to a mask frame or to a stowage zone. Hook density may be near 300 hooks cm⁻², and hook height may be about 0.25 mm, optimized to deliver peel forces above 1.4 N cm⁻¹ while remaining supple enough to contour around curved frame geometries such as forehead bars or nasal-bridge struts.

In many configurations, the repositionability illustrated by anchor patch 830 may allow each strap's proximal anchorage to migrate circumferentially in increments of roughly 30° around the bonnet, thus accommodating a broad range of mask designs. For example, when a user switches from a full-face mask having a high forehead bar to a nasal-pillow interface whose frame sits lower on the face, the user may peel top strap 810 away from anchor patch 830, rotate the strap slightly clockwise, and re-engage the hook segment onto an alternate anchor patch situated nearer the temporal region. This adjustability may eliminate the need for multiple cap SKUs tailored to specific PAP-mask geometries.

In certain embodiments, anchor patch 830 may double as a stowage zone. When top strap 810 is not needed—such as during hair-conditioning nights—the user may fold the strap backward and press distal hook-and-loop pad 820 onto anchor patch 830, thereby allowing the strap to lie flush against the shell and preventing snagging on bedding or tubing. To facilitate intuitive stowage, contrasting color bars may be screen-printed on anchor patches that serve both anchor and stowage roles, while anchor-only patches may remain monochrome; such indicia may remain legible after at least twenty domestic wash cycles due to color-fast sublimation dyes.

In other aspects, anchor patch 830 may contain an embedded neodymium disk 8 mm in diameter and 1 mm thick positioned beneath the loop pile. Distal hook-and-loop pad 820 may include a thin ferromagnetic shim laminated behind the hook substrate, creating a magnetic assist that increases normal force when the pad is pressed onto the patch. Shear-force testing may demonstrate a 12% increase in ultimate slip resistance versus hook-and-loop engagement alone, particularly beneficial in humid conditions where loop pile may soften.

In several implementations, the hook segment at the proximal end of top strap 810 may be die-cut into a cruciform shape so that four small hook “petals” can flex independently, conforming over the underlying pleat ridges of the turban-style shell and thereby maximizing effective contact area on uneven surfaces. Peel-strength tests per ASTM D5170 may show that the cruciform hook design retains at least 95% of initial engagement strength after 1 000 peel cycles, indicating long-term durability commensurate with nightly reconfiguration if desired.

In many embodiments, distal hook-and-loop pad 820 may carry a micro-thin RFID tag laminated behind the hook fabric. When the pad engages loop fabric that has an antenna trace screen-printed with conductive silver ink, the RFID tag may receive inductive power and broadcast a unique identifier. A CPAP device with an embedded reader may detect strap-engagement status, enabling compliance dashboards to record whether all required straps were fastened during therapy sessions. The antenna trace ink may weigh less than 0.5 g and may remain conductive after at least ten wash cycles.

In other configurations, top strap 810 may incorporate a low-durometer silicone strip co-extruded along its underside, increasing friction against the bonnet shell when strap tension is applied. The silicone strip thickness may be only 0.3 mm, adding negligible weight yet boosting strap-to-fabric friction coefficients by 0.15. Such friction-boosting technology may further stabilize the cap-and-mask system during side-sleep positions where gravity acts laterally on the mask frame.

In some aspects, repositionable anchor patch 830 may be laser-cut into a diamond orientation, effectively aligning the weave bias of loop fabric with dominant strap-tension vectors, thereby reducing edge curl when strap tension is high. A small bar-tack reinforcement may secure each diamond point so that shear forces distribute evenly across the patch footprint. Thread used for these bar-tacks may be aramid-blended to resist abrasion, ensuring that loop pile remains intact after repeated hook engagement.

In several embodiments, multiple anchor patches identical to repositionable anchor patch 830 may be spaced every 45 mm along pleat valleys, forming a coarse polar grid. Each grid point may be assigned a laser-etched numeral so that quick-start diagrams supplied with the product can instruct users which grid points to use for popular PAP masks—e.g., “grid point 3 for nasal-pillow masks; grid point 1 for full-face masks.” By enabling such modular spatial referencing, manufacturing can supply a single bonnet variant that accommodates diverse mask geometries and hair volumes without bespoke tailoring.

In many configurations, distal hook-and-loop pad 820 may also interface with strap-bundling clips located on the air-delivery hose so that hose weight is transferred to the cap rather than the mask frame, thereby preserving cushion seal integrity. When the user disengages pad 820 from the hose clip, a concealed loop patch on the clip's underside may provide a secondary stowage site, demonstrating that distal hook-and-loop pad 820 fulfils multiple functional roles—mask anchoring, strap stowage, and hose management.

FIGS. 9A-9C depict an operational sequence for donning the cap, routing a strap, and fastening a PAP mask frame, as shown in some embodiments.

Referring collectively to FIGS. 9A-9C, a sequential workflow 900 may be depicted that illustrates an exemplary donning procedure for a protective headgear apparatus and its cooperative interaction with a positive-airway-pressure interface. In certain aspects, FIG. 9A shows an initial positioning step in which a user grasps opposing lateral portions of a protective cap 130 and lowers the bonnet over the crown so that a dual-layer cap wall fully envelops scalp and hair. A lightly tapered lower margin hints at an internal adjustable perimeter band, which may include an elastic segment and a cinch element concealed near the nape. Once cap 130 is seated, the user may pull the cinch element in a single-handed motion, thereby decreasing cap circumference by at least five percent relative to the un-cinched state; this tension may conform the shell to the parietal vault while distributing compressive forces evenly along the hairline. Because the inner liner may be formed of satin, bamboo-rayon knit, or another low-friction textile, hair strands may slide with minimal shear during this tightening action, and scalp irritation may be mitigated.

In FIG. 9B, the workflow may progress to an alignment stage in which one of a plurality of elongate attachment straps 140 is routed forward along the temple. The illustrated strap may represent either a lateral or rear-diagonal element from the five-strap anchor system. A proximal end of strap 140 may be permanently sewn into a peripheral seam of the cap wall, whereas a distal region may carry a hook-fabric patch configured to mate with complementary loop fabric on a mask frame or a loop-equipped stowage zone. The user may hold strap 140 lightly taut so that the strap's longitudinal axis aligns with a predefined loop target on a future mask-frame engagement site. Indicia printed on the outer shell—such as color-coded arrows or numerals—may guide this alignment. Where hair volume requires strap-emergence angle modification, the user may peel the strap's proximal hook tab from a repositionable anchor patch and re-dock it at an alternate anchor patch without needlework.

In various aspects, the user may repeat the alignment procedure for remaining straps while deciding which subset best suits a selected PAP-mask style. Any straps deemed unnecessary may be folded back and pressed onto loop-fabric stowage patches sewn flush to the shell exterior so that unused straps lie flat. In one representative configuration, only the crown strap and two rear-diagonal straps may be selected for a nasal-pillow interface, whereas full five-strap deployment may be preferred for a high-profile full-face mask. Throughout alignment, strap widths may remain between 5 mm and 25 mm, and unselected straps may not interfere with the adjustable perimeter band or with volumetric pleats of a crown-volume pocket.

FIG. 9C may depict a final fastening stage in which a PAP-mask assembly—including mask external frame 229 and factory headgear—has been superimposed over cap 130. Distal hook-fabric patches at the ends of selected straps 140 may be wrapped over corresponding loop regions on frame 229, then pressed firmly to create shear-resistant joints. When strap tension is applied, the high-friction textile outer shell of cap 130 may cooperate with the taut straps to generate a distributed normal force that clamps both cap and mask against the scalp. This cooperative restraint may reduce displacement of the cushion relative to facial landmarks during neck rotation, while the low-friction inner liner simultaneously reduces frictional interaction with hair, thereby addressing twin goals of seal integrity and hair protection.

In some embodiments, the distal hook patch of a crown strap may include a micro-RFID tag laminated beneath the hook layer. When the patch contacts a loop-fabric target on frame 229, the RFID tag may inductively couple with an antenna trace embedded in the frame, signaling engagement status to a CPAP blower that records compliance metrics. Optional electronics remain water-resistant because hook-pad lamination may employ a thermoplastic polyurethane film encapsulating the tag.

In many configurations, the user may conclude the sequence by routing an air-delivery hose downward or over the head, clipping the hose to a loop sewn onto one of the rear-diagonal straps. If aromatherapy capability is desired, a diffuser disc pre-infused with lavender oil may be inserted into a pocket near the occipital seam so that volatile compounds diffuse through micro-perforations in the shell without contacting hair.

Following nightly therapy, strap disengagement may proceed in reverse order. Distal hook patches may be peeled off frame 229 and lightly pressed onto nearby stowage zones to keep straps organized during laundering. Cap 130 may then be machine-washed; hook patches sewn with ultrasonic welding may retain ≥90% peel strength after at least thirty wash cycles.

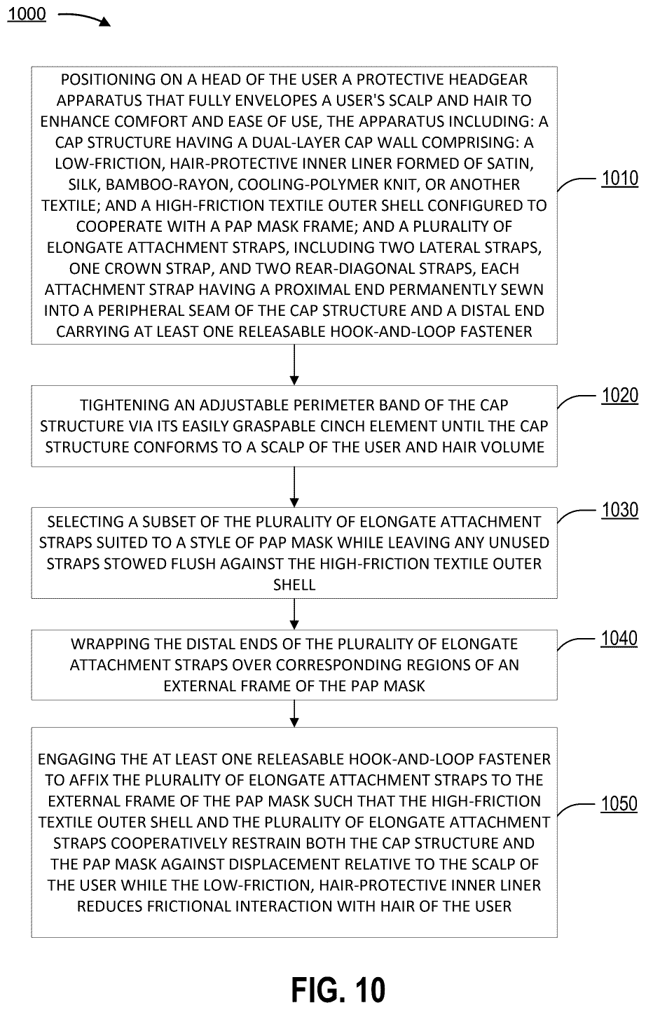

FIG. 10 depicts a flowchart outlining a method for securing a PAP mask using the protective headgear apparatus, as shown in some embodiments.

Referring to FIG. 10, a method flowchart 1000 may depict an example method for securing a positive-airway-pressure mask with a protective headgear apparatus. Rectangular blocks labelled Step 1010, Step 1020, Step 1030, Step 1040, and Step 1050 outline a sequence that, in some aspects, may be performed each evening by a therapy user or caregiver; however, other step orders, additional sub-steps, or omitted actions may also fall within the scope of the disclosed methodology.

As shown in Step 1010, the method flowchart 1000 may include positioning a protective headgear apparatus on a user's head. The apparatus may encompass a cap structure whose dual-layer wall includes a low-friction, hair-protective inner liner—such as satin, silk, bamboo-rayon knit, or cooling-polymer jersey—and a high-friction textile outer shell configured to cooperate with an external frame of a PAP mask. During positioning, the cap structure may be expanded slightly so that it fully envelopes the scalp, ears, and any high-volume hairstyles (for example, buns or locs); pleated crown-volume pockets or turban-style helices described elsewhere may accommodate such volume without scalp compression. A plurality of elongate attachment straps, including two lateral straps, one crown strap, and two rear-diagonal straps, may already reside in a stowed state—each proximal end permanently sewn into a peripheral seam of the cap wall while distal portions lie flush against exterior stowage zones.

As illustrated in Step 1020, the method may further include tightening an adjustable perimeter band via a cinch element that is easily graspable with one hand. The perimeter band may include an elastic segment joined end-to-end with a non-elastic strap routed through a ladder-lock, cam buckle, or spring-loaded cord lock. Sliding a cinch ring toward the nape region may reduce effective cap circumference by at least five percent relative to an un-cinched state, thereby conforming the outer shell to the scalp and displacing excess air gaps. In some aspects, the tightening motion may also preload the high-friction textile outer shell against hair, increasing frictional coupling so that later strap tension more effectively restrains the mask frame.

As depicted in Step 1030, the method may include selecting a subset of elongate attachment straps suited to a particular PAP-mask style while leaving any unused straps stowed flush. For example, when a nasal-pillow interface is utilized, only the crown strap and rear-diagonal straps may be necessary; lateral straps may remain folded back onto dedicated stowage patches. The selection process may be guided by indicia printed on the outer shell-color-coding might indicate preferred mask-frame targets (“blue for superior bar, green for zygomatic arms,” for example). If hair volume or mask geometry changes, the user may detach a strap's proximal hook tab from a repositionable anchor patch and press it onto a new anchor patch located at a different pleat valley.

As shown in Step 1040, the method may continue with wrapping distal ends of the selected attachment straps over corresponding regions of an external frame of the PAP mask. Strap widths may range from about 5 mm to 25 mm, and strap length may be pre-cut to span the distance between its anchor seam and a target frame bar without excess slack. Each distal segment may carry a hook-fabric pad sized approximately 40 mm×25 mm, although other dimensions may be acceptable. The user may route the crown strap over a superior frame segment, lateral straps (if employed) over cheek bars, and rear-diagonal straps beneath the coupling elbow or around posterior frame tabs. The high-friction character of the outer shell may cooperate with strap tension to generate normal forces that inhibit frame translation in sagittal, coronal, and transverse planes.

As illustrated in Step 1050, the user may engage each distal releasable fastener—typically hook-and-loop—by pressing the hook pad firmly against complementary loop fabric integrated into the mask frame or bonded to a loop tab. Engagement may also be accomplished by snap buttons, magnetic couplings, hook clasps, or combinations thereof, depending on frame construction and user dexterity. When all chosen straps are secured, the high-friction textile outer shell in concert with the taut straps may cooperatively restrain both the cap structure and the PAP mask against displacement relative to the scalp. Simultaneously, the low-friction inner liner may reduce shear stress on hair shafts, mitigating cuticular damage over repeated sleep cycles. If optional compliance electronics are integrated into distal hook pads, the final press-down action may close an inductive circuit that logs “strap engaged” status for subsequent therapy-adherence reporting.

In some implementations, a check-step may follow Step 1050, in which the user or a sensor verifies that mask leak rate and strap tension fall within acceptable ranges. Adjustments to the cinch element or strap routing may then be performed before initiating airflow therapy, although this check is omitted in the illustrated minimal sequence for clarity.

In certain aspects, the integrated system may further include a CPAP-compatible eyemask configured to overlie both eyes of a wearer while cooperating with standard CPAP headgear. The eyemask may comprise a mask body sized to span the frontal bone laterally from temple-to-temple and vertically from the supra-orbital ridge to the malar crest so that complete light blocking is achieved without impinging on the nasal bridge or disturbing the seal of a contiguous PAP mask cushion. The mask body may be fabricated as a dual-layer laminate in which an inward-facing comfort liner may include satin, bamboo-rayon knit, silk charmeuse, or cooling-microfiber filament, whereas an outward-facing light-blocking shell may utilize a densely woven microfiber twill or an opaque spacer knit. Total fabric areal density may range between 120 g m⁻² and 200 g m⁻², enabling a drape that conforms to orbital contours while remaining machine-washable for at least twenty cycles without delamination.

In some embodiments, at least one strap-interface element may be formed as a textile tunnel extending from a first side edge to a second side edge of the mask body. The tunnel may define a passage dimensioned to receive a CPAP headgear strap—notably the superior elastic band common to four-point mask harnesses. When the headgear strap is threaded through the tunnel, the strap may press the mask body against the face, thereby inhibiting displacement of the CPAP mask seal even when facial muscles relax during REM sleep. Alternative strap-interface elements may include discrete magnetic tabs or hook-and-loop loops aligned with factory headgear routing paths so that retro-fit installation requires no user sewing.

In various aspects, the eyemask may incorporate a short-range wireless audio subsystem that includes a low-energy transceiver compliant with version 5.3, a rechargeable and interconnected Li-polymer power source, and a pair of flat MEMS speakers laminated into pockets proximate opposite ear regions. The audio subsystem may reside on a detachable carrier that snaps into receptacles along the superior mask edge; removal of the carrier may permit machine laundering of the textile shell while safeguarding electronics. The carrier thickness may be <3 mm so that mask-to-headgear clearance is not compromised, and playback time may reach at least eight hours between USB-C charge cycles, thereby covering typical sleep durations. In some further aspects, a detachable electronics module may be formed by one or more of these electronic subsystems disclosed herein. This convenience may allow for removability and ability to be reinstalled, such as after a mask body was washed and dried.

In some implementations, the mask body may further include flexible earbud guide pins with corresponding grommets sewn into lateral edges. Each guide may define an elastic recess that receives a user-supplied wireless earbud, exerting a retention force of 10-50 g so that earbuds remain seated when the wearer turns laterally. Where earbuds are not used, the guides may lie flat and present no perceptible pressure.

In other configurations, an aromatherapy reservoir may be integrated along the inferior edge of the mask body. The reservoir may accept a 25 mm×40 mm felt insert infused with essential oils (e.g., lavender at 2% w/w). A hook-and-loop flap may secure the insert, allowing weekly replacement. Because the reservoir is positioned below the infra-orbital ridge, diffused vapors may travel toward nasal airways without interfering with ocular comfort.

In several embodiments, a thermally conductive insert containing phase-change microcapsules filled with n-octadecane may be laminated over the forehead region so that latent-heat absorption dampens nocturnal temperature spikes. Microcapsule diameter may average 5 μm, the total enthalpy may be about 75 J g⁻¹, and encapsulant shells may survive at least twenty wash cycles. A thin graphene-impregnated interfacing layer beneath insert may spread heat laterally, preventing localized hot spots.

To facilitate manufacturing, a method of producing eyemask may include cutting an inner liner panel and an outer shell panel to complementary contoured profiles, seaming them along their peripheries, and sewing tunnel by folding an upper edge and executing a 3-mm cover-stitch pass. Prior to final perimeter binding, the audio subsystem may be slid into a zipper-backed welt so that repair or upgrade remains user-accessible. Where a magnetic hook tab is preferred over loop fabric, a neodymium disk 10 mm in diameter and 1 mm thick may be encapsulated in TPU and bar-tacked to the mask body so that magnetic coupling with ferromagnetic inserts in CPAP headgear straps is achieved.

During use, the wearer may execute the following additional steps after donning the bonnet and fastening PAP mask straps (see Step 1050): the wearer may position the eyemask so that a tunnel aligns with the superior headgear strap, thread the strap through, and tension lightly until light leakage ceases. If the audio subsystem is present, the wearer may long-press a tactile switch to power on, then pair over Bluetooth-Low-Energy to a mobile device. Subsequent laundering may involve unsnapping carrier, removing earbud guides 930 if modular, and washing the textile components in a gentle cycle; hook integrity testing shows loop pile retains ≥90% engagement strength after twenty cycles.

In some examples, the short-range wireless transceiver complies with low-energy short-range wireless standard version 5.3 and may provide a latency of no greater than 40 ms during audio streaming. In other aspects, a magnetic tab of the eyemask may contain a neodymium magnet encased in a polymer housing that is oriented to automatically register with a ferromagnetic buckle embedded in the CPAP headgear strap. In still other examples, the earbud guides may further comprise an elastomeric retention ring that exerts a normal force of 10-50 grams on an inserted wireless earbud. In other aspects, laser-perforating the outer shell panel may be accomplished with an array of 0.25-mm micro-vents arranged outside a lock-blocking core region.

In some examples, displaying on the mobile device may include a compliance dashboard that synchronizes CPAP airflow data in response to a detected airflow pressure delivered by the CPAP mask and eyemask usage data. In other aspects, each bonnet attachment strap may include a distal or dome fastener configured to couple to the CPAP headgear, and the eyemask may be attached to the protective bonnet via a retractable hinge formed out of a shape-memory alloy ribbon.

In some examples, the eyemask body may include an edge-lit electroluminescent strip configured to emit a wake-up light pulse at a scheduled time communicated over a short-range wireless link. In other aspects, the eyemask may further comprise a short-range wireless audio subsystem wirelessly paired to a mobile computing device, and a bone-conduction transducer positioned on a temple region of the mask body.

By integrating the eyemask with the protective bonnet and CPAP headgear, the combined sleep-therapy apparatus may simultaneously address photic disturbance, hair protection, mask stabilization, audio relaxation, temperature regulation, and aromatherapy, thereby enhancing overall therapy compliance and user comfort without introducing additional headgear straps or occluding the PAP airflow conduit.

### CONCLUSION

For clarity of explanation, the above description has focused on a representative sample of all possible embodiments, a sample that teaches the principles of the invention and conveys the best mode contemplated for carrying it out. The invention is not limited to the described embodiments. Well known features may not have been described in detail to avoid unnecessarily obscuring the principles relevant to the claimed invention. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed alternatives, variations, modifications, and equivalents are within the literal scope of the following claims, and others are equivalent. The claims may be practiced without some or all of the specific details described in the specification. In many cases, method steps described in this specification can be performed in different orders than that presented in this specification, or in parallel rather than sequentially, or in different computers of a computer network, rather than all on a single computer. It is to be understood that the present technology contemplates that, to the extent possible, one or more features of any implementation can be combined with one or more features of any other implementation.

While the above description details certain embodiments of the invention and describes the best mode contemplated, no matter how detailed the above appears in text, the invention can be practiced in many ways. Therefore, implementation details may vary considerably while still being encompassed by the invention disclosed herein. Particular terminology used when describing certain features or aspects of the invention should not be taken to imply that the terminology is being redefined herein to be restricted to any specific characteristics, features, or aspects of the invention with which that terminology is associated.

What is claimed is:

1. A universal protective headgear apparatus configured for use with a positive-airway-pressure mask (PAP mask), including, but not limited to, Continuous Positive Airway Pressure (CPAP), Bi-level PAP (BiPAP), Automatic PAP (APAP) masks, comprising:
a flexible, breathable cap structure dimensioned to cover a wearer's scalp and hair for enhanced comfort and comprehensive hair protection, the cap structure having an interior surface that is hair-protective and an adjustable perimeter band including an elastic segment and cinch element configured for quick, one-handed adjustment; and
a plurality of elongate attachment straps, each attachment strap having a proximal end affixed to the cap structure and a distal end carrying a releasable fastener;
wherein the plurality of elongate attachment straps are dimensioned and positioned such that, when the cap structure is donned and the distal ends of the plurality of elongate attachment straps are wrapped over an external frame of the universal protective headgear and secured via releasable fasteners, an exterior, mask-facing surface of the cap structure together with the plurality of elongate attachment straps cooperatively restrain both the cap structure and the PAP mask against displacement relative to the wearer's scalp, while the interior surface of the cap structure reduces frictional interaction with the wearer's hair.

2. The protective headgear apparatus of claim 1, wherein the plurality of elongate attachment straps comprises at least five straps including two lateral straps, one crown strap, and two diagonal rear straps, thereby providing 360-degree stabilization.

3. The protective headgear apparatus of claim 1, wherein the releasable fasteners comprise hook-and-loop, buttons, hooks, or similar material.

4. The protective headgear apparatus of claim 1, wherein the proximal end of each attachment strap is permanently sewn into a seam of the cap structure.

5. The protective headgear apparatus of claim 1, further comprising one or more stowage zones on an exterior surface of the cap structure, each stowage zone being configured to releasably engage a distal fastener of an unused one of the plurality of elongate attachment straps so that an unused strap lies flush against the cap structure when not required for a given PAP-mask style.

6. The protective headgear apparatus of claim 1, wherein the adjustable perimeter band is dimensioned such that activation of the cinch element decreases a circumference of the cap structure by at least five percent relative to an un-cinched state.

7. The protective headgear apparatus of claim 1, wherein the plurality of elongate attachment straps is repositionable by way of mating hook-and-loop anchor patches or a snap-button grid disposed on the cap structure.

8. The protective headgear apparatus of claim 1, wherein each attachment strap has a width between 5 mm and 25 mm and a length sufficient to wrap over a frame portion of a positive-airway-pressure (PAP) mask, including but not limited to a forehead bar of a nasal pillow, nasal, or full-face mask.

9. The protective headgear apparatus of claim 1, further comprising indicia printed on the cap structure to guide user alignment of the plurality of elongate attachment straps with predefined regions of the PAP mask.

10. The protective headgear apparatus of claim 1, wherein the cap structure is fabricated from a flexible, machine-washable material able to withstand at least thirty laundering cycles without degradation of the releasable fasteners.

11. The protective headgear apparatus of claim 1, wherein the releasable fasteners are further selected from buttons, snaps, hook clasps, magnetic couplings, or combinations thereof.

12. A method of securing a positive-airway-pressure (PAP) therapy mask, including CPAP, BiPAP, or APAP masks, to a user, the method comprising:
positioning on a head of the user a protective headgear apparatus that fully envelopes a user's scalp and hair to enhance comfort and ease of use, the apparatus including:
a cap structure having a dual-layer cap wall comprising:
a low-friction, hair-protective inner liner formed of satin, silk, bamboo-rayon, cooling-polymer knit, or another textile; and
a high-friction textile outer shell configured to cooperate with a PAP mask frame; and
a plurality of elongate attachment straps, including two lateral straps, one crown strap, and two rear-diagonal straps, each attachment strap having a proximal end permanently sewn into a peripheral seam of the cap structure and a distal end carrying at least one releasable hook-and-loop fastener;
tightening an adjustable perimeter band of the cap structure via its easily graspable cinch element until the cap structure conforms to a scalp of the user and hair volume;
selecting a subset of the plurality of elongate attachment straps suited to a style of PAP mask while leaving any unused straps stowed flush against the high-friction textile outer shell;
wrapping the distal ends of the plurality of elongate attachment straps over corresponding regions of an external frame of the PAP mask; and
engaging the at least one releasable hook-and-loop fastener to affix the plurality of elongate attachment straps to the external frame of the PAP mask such that the high-friction textile outer shell and the plurality of elongate attachment straps cooperatively restrain both the cap structure and the PAP mask against displacement relative to the scalp of the user while the low-friction, hair-protective inner liner reduces frictional interaction with hair of the user.

13. The method of claim 12, further comprising sliding a cinch ring along an elastic segment of the adjustable perimeter band to reduce an effective circumference of the cap structure by at least five percent.

14. The method of claim 12, further comprising stowing any unused ones of the plurality of elongate attachment straps against an exterior surface of the cap structure by releasably engaging their hook-and-loop fasteners.

15. The method of claim 12, further comprising positioning an aromatherapy element on the headgear apparatus, the aromatherapy element selected from:
a removable or replaceable fabric insert infused with essential oils;
a diffuser tab or disc retained by a pocket or clip on the cap structure; or
a region of the cap structure pre-treated with essential oils configured to release scent over time.

16. The method of claim 12, further comprising detaching at least one of the plurality of elongate attachment straps from a first anchor region of the cap structure and repositioning the plurality of elongate attachment straps onto a second, different anchor region of the cap structure to accommodate a change in a volume of hair of the user.

17. A method of manufacturing a protective headgear apparatus configured for use with a positive-airway-pressure (PAP) therapy mask, including CPAP, BiPAP, or APAP masks, the method comprising:
cutting a first textile panel and a second textile panel to complementary bonnet profiles;
joining the first textile panel and the second textile panel along a peripheral seam to form a cap wall;
sewing proximal ends of a plurality of attachment straps into the peripheral seam;
affixing at least one releasable fastener to each distal end of the plurality of attachment straps; and
orienting the plurality of attachment straps such that, when a cap structure is donned and distal ends of the plurality of attachment straps are wrapped over an external frame of a PAP mask and secured via the at least one releasable fastener, the cap structure and the PAP mask are restrained against displacement relative to a wearer's scalp.

18. The method of claim 17, further comprising ultrasonically bonding hook-and-loop material to the distal ends of the plurality of attachment straps.

19. The method of claim 17, further comprising orienting the first textile panel as an inner satin liner and the second textile panel as an outer polyester-cotton blend shell.

20. The method of claim 17, further comprising installing a matrix of complementary hook-and-loop anchor patches on an exterior surface of the cap wall to enable later repositioning of the plurality of attachment straps.
